(19) United States
(12) Patent Application Publication
(10) Pub. No.: US 2024/0127486 A1
(43) Pub. Date: Apr. 18, 2024

(54) WALKING INDEX CALCULATION DEVICE, WALKING INDEX CALCULATION SYSTEM, WALKING INDEX CALCULATION METHOD, AND PROGRAM RECORDING MEDIUM

(71) Applicant: NEC, Corporation, Tokyo (JP)

(72) Inventors: Chenhui HUANG, Tokyo (JP); Fumiyuki NIHEY, Tokyo (JP); Kenichiro FUKUSHI, Tokyo (JP); Zhenwei WANG, Tokyo (JP)

(73) Assignee: NEC Corporation, Tokyo (JP)

(21) Appl. No.: 18/396,112

(22) Filed: Dec. 26, 2023

Related U.S. Application Data
(63) Continuation of application No. 18/035,328, filed on May 4, 2023.

(51) Int. Cl. G06T 9/00; G06V 10/75

(52) U.S. Cl. CPC G06T 9/00; G06V 10/751

(57) ABSTRACT

A walking index calculation device that includes a waveform generation unit configured to generate a walking waveform by using sensor data regarding motion of a foot acquired by a sensor installed in footwear; a detection unit configured to detect a timing at which a clearance of a toe is minimized from the walking waveform; and a calculation unit configured to calculate a minimum value of the clearance of the toe by using a walking parameter at the timing at which the clearance of the toe is minimized.

This application is a Continuation of U.S. application Ser. No. 18/038,328 filed on May 23, 2023, which is a National Stage Entry of PCT/JP2020/044722 filed on Dec. 1, 2020, the contents of all of which are incorporated herein by reference, in their entirety.

### TECHNICAL FIELD

The present disclosure relates to a walking index calculation device or the like that calculates an index regarding walking.

### BACKGROUND ART

With increasing interest in healthcare that performs physical condition management, attention has been focused on a service that measures a gait including a walking feature and provides information corresponding to the gait to a user. For example, a device in which an inertial measurement device is mounted on footwear such as shoes and that analyzes a gait of a user has been developed. For example, if the clearance of the toe of the user can be analyzed in daily life, there is a possibility that the risk of falling or the like during walking can be reduced.

PTL 1 discloses a tripping risk evaluation device that evaluates a tripping risk. The device in PTL 1 calculates the clearance of the toe from at least one of left/right component data, vertical component data, and forward/backward component data of the floor reaction force, based on floor reaction force data indicating a change in the floor reaction force in a walking action. The device in PTL 1 evaluates a tripping risk based on the calculated clearance of the toe.

PTL 2 discloses a technique for extracting parameters related to motion of a leg during walking based on motion due to a predetermined action in a cooperation part that moves in cooperation with the leg. In the technique in PTL 2, time-series first data obtained from a sensor that measures motion of a leg during walking is measured. In the technique in PTL 2, time-series second data obtained from a sensor that measures motion due to a predetermined action in a cooperation part that moves in cooperation with a leg is measured. In the technique in PTL 2, a conversion system for converting the first data is determined in such a way that the similarity between the first data and the second data is maximized, and the first data is converted based on the determined conversion system.

PTL 3 discloses a walking speed detection device that detects a walking speed of a wearer. The device in PTL 3 calculates a walking speed by using acceleration detected by a biaxial acceleration detection sensor and angle amplitude data for the foot during walking recorded in advance.

NPL 1 discloses a method of estimating a foot clearance by using a wireless inertial sensor system attached to a foot. In the method in NPL 1, sensor signal data is fused to calculate an orientation and a trajectory of the foot, and timings of toe off and heel strike are detected. In the method in NPL 1, positions of sensors with respect to trajectories of the foot, the heel, and the toe are estimated based on a kinematic model based on the detected timings of toe off and heel strike. In the method in NPL 1, parameters related to the minimum value and the maximum value of a clearance of the heel and the toe are extracted based on the positions of the sensors with respect to the estimated trajectories of the foot, the heel, and the toe.

### CITATION LIST

### Patent Literature

PTL 1: Japanese Patent No. 5915990
PTL 2: WO 2017/179090 A1
PTL 3: JP 2005-233771 A

### Non Patent Literature

NPL 1: Benoit Mariani, Stephane Rochat, Christophe J. Bula, Kamiar Aminian, “Heel and Toe Clearance Estimation for Gait Analysis Using Wireless Inertial Sensors”, IEEE Transactions on Biomedical Engineering, Volume 59, Issue 11, pp. 3162-3168.

### SUMMARY OF INVENTION

### Technical Problem

In the method in PTL 1, it is necessary to measure the floor reaction force in order to calculate the clearance of the toe. Since it is difficult to mount a sensor that measures the floor reaction force on footwear such as shoes, it is difficult to apply the method in PTL 1 to daily life.

In the method in PTL 2, when detecting/quantifying walking by using a pattern of a sensor waveform, a speed, a timing, and the like of stepping out, landing, and release are extracted by assuming a rotation speed in a front-back direction during walking. In the method in PTL 2, speeds, timings, and the like of stepping out, landing, and release are extracted, but the clearance of the toe cannot be estimated with only these parameters.

In the method in PTL 3, a walking speed can be detected. However, in the method in PTL 3, the clearance of the foot cannot be evaluated.

In the method in NPL 1, it is necessary to calculate all trajectories of the foot, the heel, and the toe. Since the method in NPL 1 requires a large amount of calculation, it is difficult to apply the method to the calculation of the clearance of the toe in daily life.

An object of the present disclosure is to provide a walking index calculation device and the like capable of calculating a clearance of a toe during walking in daily life.

### Solution to Problem

A walking index calculation device according to an aspect of the present disclosure includes a waveform generation unit configured to generate a walking waveform by using sensor data regarding motion of a foot acquired by a sensor installed in footwear; a detection unit configured to detect a timing at which a clearance of a toe is minimized from the walking waveform; and a calculation unit configured to calculate a minimum value of the clearance of the toe by using a walking parameter at the timing at which the clearance of the toe is minimized.

A walking index calculation method according to an aspect of the present disclosure includes causing a computer to generate a walking waveform by using sensor data regarding motion of a foot acquired by a sensor installed in footwear; detect a timing at which a clearance of a toe is minimized from the walking waveform; and calculate a minimum value of the clearance of the toe by using a walking parameter at the timing at which the clearance of the toe is minimized.

A program according to an aspect of the present disclosure causes a computer to execute a process of generating a walking waveform by using sensor data regarding motion of a foot acquired by a sensor installed in footwear; a process of detecting a timing at which a clearance of a toe is minimized from the walking waveform; and a process of calculating a minimum value of the clearance of the toe by using a walking parameter at the timing at which the clearance of the toe is minimized.

### Advantageous Effects of Invention

According to the present disclosure, it is possible to provide a walking index calculation device and the like capable of calculating a clearance of a toe in walking in daily life.

### BRIEF DESCRIPTION OF DRAWINGS

FIG. 1 is a block diagram illustrating an example of a configuration of a walking index calculation system according to a first example embodiment.

FIG. 2 is a conceptual diagram illustrating a disposition example of a data acquisition device of the walking index calculation system according to the first example embodiment.

FIG. 3 is a conceptual diagram for describing a coordinate system set in the data acquisition device of the walking index calculation system according to the first example embodiment.

FIG. 4 is a conceptual diagram for describing a human body surface applied to a walking index calculation device of the walking index calculation system according to the first example embodiment.

FIG. 5 is a conceptual diagram for describing a walking event.

FIG. 6 is a conceptual diagram for describing a minimum toe clearance (MTC).

FIG. 7 is a conceptual diagram for describing a timing of an MTC in a trajectory of a toe height.

FIG. 8 is a block diagram illustrating an example of a configuration of the data acquisition device of the walking index calculation system according to the first example embodiment.

FIG. 9 is a block diagram illustrating an example of a configuration of the walking index calculation device of the walking index calculation system according to the first example embodiment.

FIG. 10 is a conceptual diagram for describing measurement of walking parameters using motion capture.

FIG. 11 is a conceptual diagram for describing disposition of cameras used for measurement of walking parameters using motion capture.

FIG. 12 is a graph illustrating an example of a trajectory of a foot measured by using motion capture.

FIG. 13 is a graph illustrating test results for a timing of foot adjacent and a timing of an MTC measured by using motion capture.

FIG. 14 is a graph for describing a timing of foot adjacent detected from a walking waveform of advancing direction acceleration generated by the walking index calculation device of the walking index calculation system according to the first example embodiment.

FIG. 15 is a graph illustrating test results for a timing (estimated value) of foot adjacent detected by the walking index calculation device of the walking index calculation system according to the first example embodiment and a timing (true value) of foot adjacent measured by using motion capture.

FIG. 16 is a graph for describing a timing of zero crossing detected from a walking waveform of a vertical acceleration generated by the walking index calculation device of the walking index calculation system according to the first example embodiment.

FIG. 17 is a graph illustrating test results for a timing of zero crossing in a walking waveform of a vertical acceleration detected by the walking index calculation device of the walking index calculation system according to the first example embodiment and a timing of an MTC measured by using motion capture.

FIG. 18 is a conceptual diagram for describing a calculation example of an MTC performed by the walking index calculation device of the walking index calculation system according to the first example embodiment.

FIG. 19 is a graph for describing a calculation example (pattern 1) of an MTC performed by the walking index calculation device of the walking index calculation system according to the first example embodiment.

FIG. 20 is a conceptual diagram for describing measurement of a true value (MTCO) of an MTC using motion capture.

FIG. 21 is a graph illustrating test results for an estimated value (pattern 1) of an MTC calculated by the walking index calculation device of the walking index calculation system according to the first example embodiment and a true value of an MTC measured by using motion capture.

FIG. 22 is a graph for describing a calculation example (pattern 2) of an MTC performed by the walking index calculation device of the walking index calculation system according to the first example embodiment.

FIG. 23 is a graph illustrating test results for an estimated value (pattern 2) of an MTC calculated by the walking index calculation device of the walking index calculation system according to the first example embodiment and a true value of an MTC measured by using motion capture.

FIG. 24 is a flowchart for describing an example of an operation of the walking index calculation device of the walking index calculation system according to the first example embodiment.

FIG. 25 is a flowchart for describing an example (pattern 1) of a walking index calculation process performed by the walking index calculation device of the walking index calculation system according to the first example embodiment.

FIG. 26 is a flowchart for describing an example (pattern 2) of a walking index calculation process performed by the walking index calculation device of the walking index calculation system according to the first example embodiment.

FIG. 27 is a block diagram illustrating an example of a configuration of a walking index calculation system according to a second example embodiment.

FIG. 28 is a block diagram illustrating an example of a configuration of a walking index calculation device of the walking index calculation system according to the second example embodiment.

FIG. 29 is a conceptual diagram for describing a calculation example of a sensor position in an advancing direction at a timing of toe off, performed by the walking index calculation device of the walking index calculation system according to the second example embodiment.

FIG. 30 is a graph for describing a calculation example of the sensor position in the advancing direction at the timing of toe off, performed by the walking index calculation device of the walking index calculation system according to the second example embodiment.

FIG. 31 is a flowchart for describing an example of a walking index calculation process performed by the walking index calculation device of the walking index calculation system according to the second example embodiment.

FIG. 32 is a block diagram illustrating an example of a configuration of a walking index calculation system according to a third example embodiment.

FIG. 33 is a block diagram illustrating an example of a configuration of a walking index calculation device of the walking index calculation system according to the third example embodiment.

FIG. 34 is a graph for describing a determination result from a determination unit of the walking index calculation device of the walking index calculation system according to the third example embodiment.

FIG. 35 is a conceptual diagram illustrating an example in which the determination result from the determination unit of the walking index calculation device of the walking index calculation system according to the third example embodiment is displayed on a display unit of a mobile terminal.

FIG. 36 is a flowchart for describing an example of a walking index calculation process performed by the walking index calculation device of the walking index calculation system according to the third example embodiment.

FIG. 37 is a block diagram illustrating an example of a configuration of a walking index calculation device according to a fourth example embodiment.

FIG. 38 is a block diagram illustrating an example of a hardware configuration for implementing the walking index calculation device according to each example embodiment.

### EXAMPLE EMBODIMENTS

Hereinafter, example embodiments of the present invention will be described with reference to the drawings. However, although the example embodiments described below have technically preferable limitations for carrying out the present invention, the scope of the invention is not limited to the following description. In all the drawings used in the following description of the example embodiments, other than a particular reason, the same reference numerals are given to the same parts. In the following example embodiments, repeated description of similar configurations and operations may be omitted.

### First Example Embodiment

First, a walking index calculation system according to a first example embodiment will be described with reference to the drawings. The walking index calculation system of the present example embodiment calculates a walking index by using a waveform (also referred to as a walking waveform) based on time-series data of sensor data acquired by a sensor installed on a foot portion of a pedestrian. The walking index calculation system of the present example embodiment calculates a clearance of a toe for each step. The clearance of the toe is a walking index for measuring to what extent the toe of the foot has a margin from the ground. In particular, the walking index calculation system of the present example embodiment calculates, as the clearance of the toe, a minimum toe clearance (MTC) in a period (swing phase) in which the foot is separated from the ground during walking.

(Configuration)

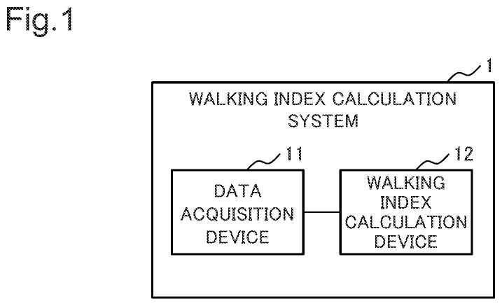

FIG. 1 is a block diagram illustrating a configuration of a walking index calculation system 1 of the present example embodiment. The walking index calculation system 1 includes a data acquisition device 11 and a walking index calculation device 12. The data acquisition device 11 and the walking index calculation device 12 may be connected by wire or wirelessly. The data acquisition device 11 and the walking index calculation device 12 may be configured by a single device. The walking index calculation system 1 may include only the walking index calculation device 12 without including the data acquisition device 11.

For example, the data acquisition device 11 is installed in footwear such as shoes. In the present example embodiment, an example in which the data acquisition device 11 is disposed at a position on the back side of the arch of the foot will be described. The data acquisition device 11 includes an acceleration sensor and an angular velocity sensor. The data acquisition device 11 measures a physical quantity related to motion of a foot such as a spatial acceleration and a spatial angular velocity as a physical quantity related to motion of a foot of a user wearing footwear. The physical quantity related to the motion of the foot measured by the data acquisition device 11 includes not only an acceleration and an angular velocity but also a velocity and an angle calculated by integrating the acceleration and the angular velocity. The physical quantity related to the motion of the foot measured by the data acquisition device 11 also includes a position (trajectory) calculated through second-order integration of acceleration.

The data acquisition device 11 converts the measured physical quantity into digital data (also referred to as sensor data). The data acquisition device 11 transmits the converted sensor data to the walking index calculation device 12. For example, the data acquisition device 11 is connected to the walking index calculation device 12 via a mobile terminal (not illustrated) carried by the user. A mobile terminal (not illustrated) is a communication device that can be carried by a user. For example, the mobile terminal is a portable communication device having a communication function, such as a smartphone, a smart watch, or a mobile phone. The mobile terminal receives, from the data acquisition device 11, the sensor data regarding the motion of the user's foot. The mobile terminal transmits the received sensor data to a server or the like on which the walking index calculation device 12 is mounted. The function of the walking index calculation device 12 may be achieved by an application installed in the mobile terminal. In that case, the mobile terminal processes the received sensor data with application software installed therein.

The data acquisition device 11 is implemented by, for example, an inertial measurement device including an acceleration sensor and an angular velocity sensor. An example of the inertial measurement device is an inertial measurement unit (IMU). The IMU includes a three-axis acceleration sensor and a three-axis angular velocity sensor. The inertial measurement device may be a vertical gyro (VG), an attitude heading (AHRS), a global positioning system/inertial navigation system (GPS/INS), or the like.

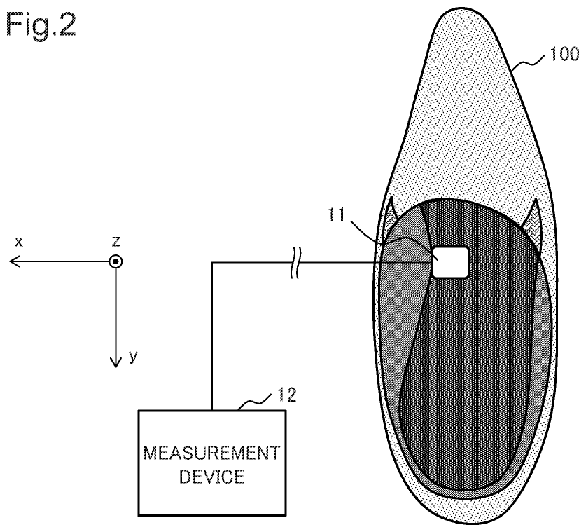

FIG. 2 is a conceptual diagram illustrating an example in which the data acquisition device 11 is installed in a shoe 100. In the example in FIG. 2, the data acquisition device 11 is installed at a position corresponding to a back side of the arch of a foot. For example, the data acquisition device 11 is installed in an insole inserted into the shoe 100. For example, the data acquisition device 11 is installed on the bottom surface of the shoe 100. For example, the data acquisition device 11 is embedded in a main body of the shoe 100. The data acquisition device 11 may be detachable from the shoe 100 or may not be detachable from the shoe 100. The data acquisition device 11 may be installed at a position that is not the back side of the arch of the foot as long as it can acquire sensor data regarding the motion of the foot. The data acquisition device 11 may be installed on a sock worn by the user or a decorative article such as an anklet worn by the user. The data acquisition device 11 may be directly attached to the foot or may be embedded in the foot. FIG. 2 illustrates an example in which the data acquisition device 11 is installed in the shoe 100 of the right foot. The data acquisition device 11 only needs to be installed on at least one foot, and may be installed on both left and right feet. If the data acquisition device 11 is installed in the shoes 100 of both feet, a walking event can be detected in association with the motion of both feet.

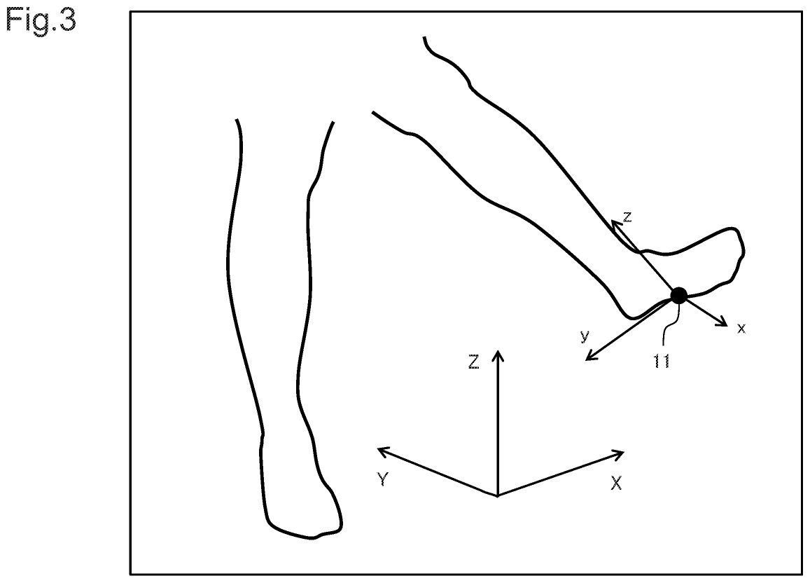

FIG. 3 is a conceptual diagram for describing a local coordinate system (x axis, y axis, z axis) set in the data acquisition device 11 and a world coordinate system (X axis, Y axis, Z axis) set for the ground in a case where the data acquisition device 11 is installed on the back side of the arch of foot. In the world coordinate system (X axis, Y axis, Z axis), in a state in which the user is standing upright, a lateral direction of the user is set to an X axis direction (a leftward orientation is positive), a direction of a back surface of the user is set to a Y axis direction (a rearward orientation is positive), and a gravity direction (also referred to as a vertical direction) is set to a Z axis direction (a vertically upward orientation is positive). In the present example embodiment, a local coordinate system including an x direction, a y direction, and a z direction based on the data acquisition device 11 is set. In the present example embodiment, a coordinate system in the same direction is used for the left and right feet.

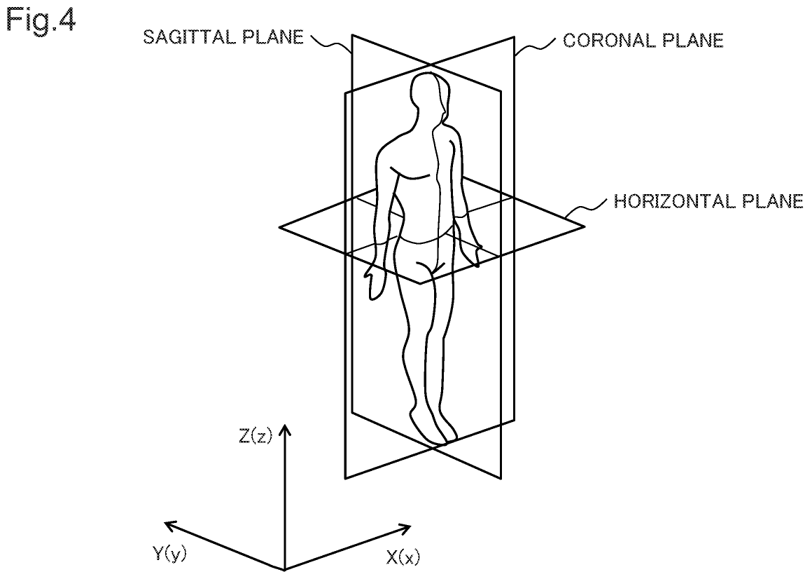

FIG. 4 is a conceptual diagram for describing a surface (also referred to as a human body surface) set for the human body. In the present example embodiment, a sagittal plane dividing the body into left and right, a coronal plane dividing the body into front and rear, and a horizontal plane dividing the body horizontally are defined. In the upright state as illustrated in FIG. 4, the world coordinate system coincides with the local coordinate system. In the present example embodiment, rotation in the sagittal plane with the x axis as a rotation axis is defined as roll, rotation in the coronal plane with the y axis as a rotation axis is defined as pitch, and rotation in the horizontal plane with the z axis as a rotation axis is defined as yaw. A rotation angle in the sagittal plane with the x axis as a rotation axis is defined as a roll angle, a rotation angle in the coronal plane with the y axis as a rotation axis is defined as a pitch angle, and a rotation angle in the horizontal plane with the z axis as a rotation axis is defined as a yaw angle. In the present example embodiment, when the body is viewed from the right side, clockwise rotation in the sagittal plane is defined as positive, and counterclockwise rotation in the sagittal plane is defined as negative.

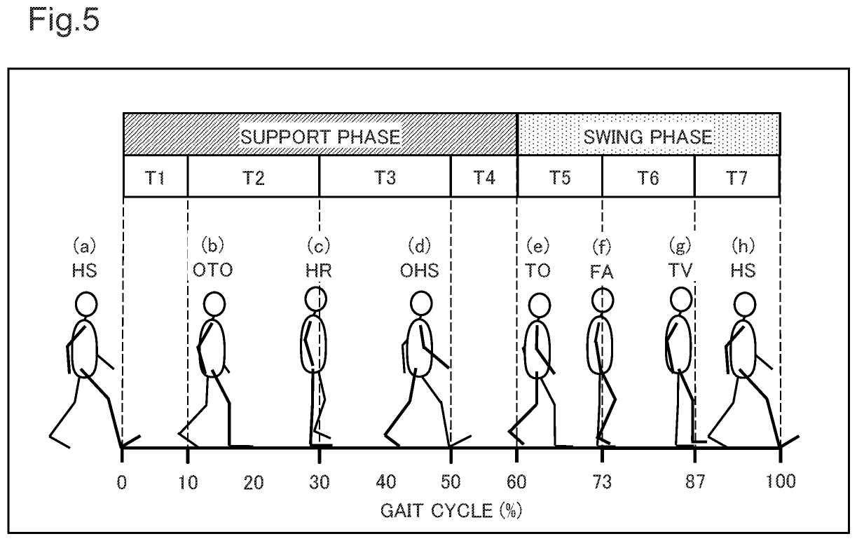

FIG. 5 is a conceptual diagram for describing one gait cycle with the right foot as a reference. One gait cycle based on the left foot is also similar to that of the right foot. The horizontal axis in FIG. 5 represents a gait cycle normalized with one gait cycle of the right foot as 100%, the one gait cycle being a cycle in which a time point at which the heel of the right foot lands on the ground as a start point and a time point at which the heel of the right foot next lands on the ground as an end point. The one gait cycle of one foot is roughly divided into a support phase in which at least a part of the back side of the foot is in contact with the ground and a swing phase in which the back side of the foot is separated from the ground. The support phase is further subdivided into a support initial stage T1, a support middle stage T2, a support end stage T3, and a swing early stage T4. The swing phase is further subdivided into a swing initial stage T5, a swing middle stage T6, and a swing end stage T7.

(a) of FIG. 5 represents an event in which the heel of the right foot comes into contact with the ground (heel strike: HS). (b) of FIG. 5 represents an event in which the toe of the left foot moves away from the ground while the sole of the right foot is in contact with the ground (opposite toe off: OTO). (c) of FIG. 5 represents an event in which the heel of the right foot rises while the sole of the right foot is in contact with the ground (heel rise: HR). (d) of FIG. 5 represents an event in which the heel of the left foot is in contact with the ground (opposite heel strike: OHS). (e) of FIG. 5 represents an event in which the toe of the right foot moves away from the ground in a state in which the sole of the left foot is in contact with the ground (toe off: TO). (f) of FIG. 5 illustrates an event in which the left foot and the right foot cross each other in a state in which the sole of the left foot is in contact with the ground (foot adjacent: FA). (g) of FIG. 5 represents an event in which the tibia of the right foot is substantially perpendicular to the ground in a state in which the sole of the left foot is in contact with the ground (tibia vertical: TV). (h) of FIG. 5 represents an event in which the heel of the right foot comes into contact with the ground (heel strike: HS). (h) of FIG. 5 corresponds to the end point of the gait cycle starting from (a) of FIG. 5 and corresponds to the start point of the next gait cycle.

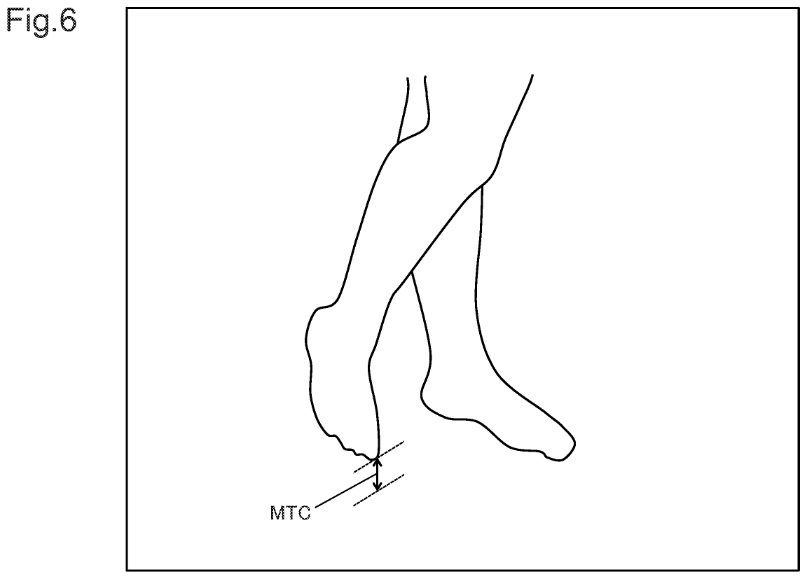

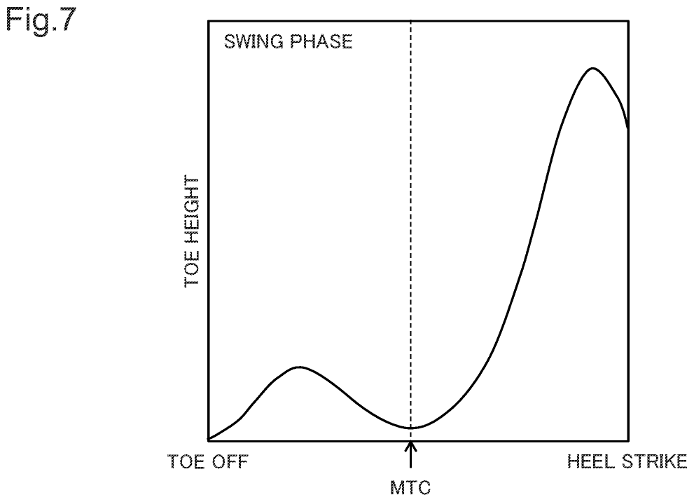

FIG. 6 is a conceptual diagram for describing the clearance of the toe. FIG. 6 illustrates a timing at which the clearance of the toe of the right foot is minimized in a period of the swing phase. In the present example embodiment, the minimum value of the clearance of the toe will be referred to as a minimum toe clearance (MTC). FIG. 7 is a graph illustrating an example of a trajectory of the toe height in a period (swing phase) from toe off to heel strike. When the MTC is small, there is a high risk of tripping even with a small step. In the present example embodiment, the MTC is calculated by using sensor data measured by the data acquisition device 11. If the MTC during walking can be calculated, information according to a value of or a change in the MTC can be provided.

The walking index calculation device 12 acquires sensor data regarding the motion of the foot of the user. The walking index calculation device 12 generates a waveform (also referred to as a walking waveform) based on time-series data of the acquired sensor data. The walking index calculation device 12 detects a timing of the MTC from the generated walking waveform. For example, the walking index calculation device 12 detects a timing of foot adjacent detected from a walking waveform of an acceleration in the Y direction (advancing direction acceleration) in the sagittal plane as a timing of the MTC (pattern 1). For example, the walking index calculation device 12 detects a timing of zero crossing detected from a walking waveform of an acceleration in the Z direction (vertical acceleration) in the sagittal plane/coronal plane as a timing of the MTC (pattern 2). The walking index calculation device 12 calculates a walking index (value of the MTC) by using values of a vertical height and a roll angle at the detected timing of the MTC. A specific MTC calculation method using the walking index calculation device 12 will be described later.

The walking index calculation device 12 outputs the calculated value of the MTC. For example, the value of the MTC output from the walking index calculation device 12 is displayed on a screen of a terminal device (not illustrated) carried by the user or a screen of a display device (not illustrated). For example, the value of the MTC output from the walking index calculation device 12 is output to a system (not illustrated) that analyzes the value of the MTC. For example, the value of the MTC output from the walking index calculation device 12 is accumulated in a database (not illustrated) and used as big data. A use of the value of the MTC output from the walking index calculation device 12 is not particularly limited.

[Data Acquisition Device]

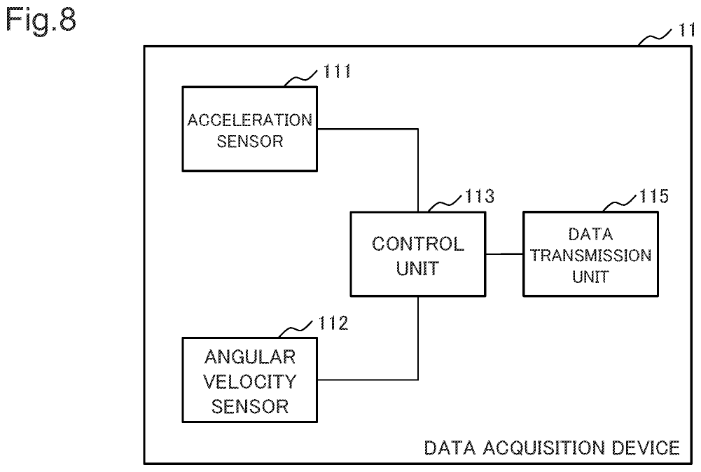

Next, details of the data acquisition device 11 will be described with reference to the drawings. FIG. 8 is a block diagram illustrating an example of a detailed configuration of the data acquisition device 11. The data acquisition device 11 includes an acceleration sensor 111, an angular velocity sensor 112, a control unit 113, and a data transmission unit 115. The data acquisition device 11 includes a power supply (not illustrated). In the following description, each of the acceleration sensor 111, the angular velocity sensor 112, the control unit 113, and the data transmission unit 115 will be described as an operation subject, but the data acquisition device 11 may be regarded as an operation subject.

The acceleration sensor 111 is a sensor that measures accelerations (also referred to as spatial accelerations) in three-axis directions. The acceleration sensor 111 outputs the measured accelerations to the control unit 113. For example, a sensor of a piezoelectric type, a piezoresistive type, a capacitance type, or the like may be used as the acceleration sensor 111. A measurement method in a sensor used for the acceleration sensor 111 is not limited as long as the sensor can measure acceleration.

The angular velocity sensor 112 is a sensor that measures angular velocities in three-axis directions (also referred to as spatial angular velocities). The angular velocity sensor 112 outputs the measured angular velocities to the control unit 113. For example, a sensor of a vibration type, a capacitance type, or the like may be used as the angular velocity sensor 112. A measurement method in a sensor used for the angular velocity sensor 112 is not limited as long as the sensor can measure an angular velocity.

The control unit 113 acquires accelerations and angular velocities in the three-axis directions from the acceleration sensor 111 and the angular velocity sensor 112, respectively. The control unit 113 converts the acquired accelerations and angular velocities into digital data, and outputs the converted digital data (also referred to as sensor data) to the data transmission unit 115. The sensor data includes at least acceleration data obtained by converting analog acceleration data into digital data and angular velocity data obtained by converting analog angular velocity data into digital data. The acceleration data includes acceleration vectors in the three-axis directions. The angular velocity data includes angular velocity vectors in the three-axis directions. Acquisition times of the acceleration data and the angular velocity data are linked to the acceleration data and the angular velocity data. The control unit 113 may be configured to output sensor data obtained by applying correction such as correction of a mounting error or a temperature, and linearity correction to the acquired acceleration data and angular velocity data. The control unit 113 may generate angle data in the three-axis directions by using the acquired acceleration data and angular velocity data.

For example, the control unit 113 is a microcomputer or a microcontroller that performs overall control and data processing of the data acquisition device 11. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure an angular velocity and acceleration. For example, the control unit 113 performs analog-to-digital conversion (AD conversion) on physical quantities (analog data) such as the measured angular velocity and acceleration, and stores the converted digital data in a flash memory. The physical quantities (analog data) measured by the acceleration sensor 111 and the angular velocity sensor 112 may be converted into digital data in the acceleration sensor 111 and the angular velocity sensor 112. The digital data stored in the flash memory is output to the data transmission unit 115 at a predetermined timing.

The data transmission unit 115 acquires sensor data from the control unit 113. The data transmission unit 115 transmits the acquired sensor data to the walking index calculation device 12. For example, the data transmission unit 115 transmits the sensor data to the walking index calculation device 12 via wireless communication. For example, the data transmission unit 115 is configured to transmit the sensor data to the walking index calculation device 12 via a wireless communication function (not illustrated) conforming to a standard such as Bluetooth (registered trademark) or WiFi (registered trademark). The communication function of the data transmission unit 115 may conform to a standard other than Bluetooth (registered trademark) or WiFi (registered trademark). For example, the data transmission unit 115 may transmit the sensor data to the walking index calculation device 12 by wire such as a cable.

[Walking Index Calculation Device]

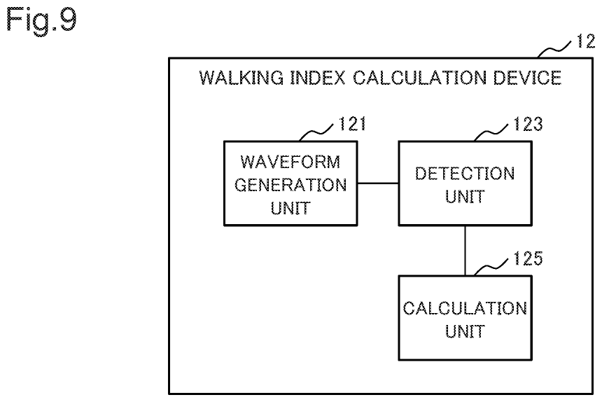

Next, details of the walking index calculation device 12 will be described with reference to the drawings. FIG. 9 is a block diagram illustrating an example of a configuration of the walking index calculation device 12. The walking index calculation device 12 includes a waveform generation unit 121, a detection unit 123, and a calculation unit 125.

The waveform generation unit 121 acquires sensor data from the data acquisition device 11 (sensor) installed on footwear worn by s pedestrian. By using the sensor data, the waveform generation unit 121 generates time-series data (also referred to as a walking waveform) associated with walking of the pedestrian wearing the footwear in which the data acquisition device 11 is installed.

For example, the waveform generation unit 121 generates a walking waveform of a spatial acceleration, a spatial angular velocity, or the like. The waveform generation unit 121 integrates the spatial acceleration or the spatial angular velocity, and generates a walking waveform of a spatial velocity, a spatial angle (plantar angle), or the like. The waveform generation unit 121 performs second-order integration of the spatial acceleration to generate a walking waveform of a spatial trajectory. The waveform generation unit 121 generates a walking waveform at a predetermined timing or a time interval set in accordance with a general gait cycle or a gait cycle unique to a user. A timing at which the waveform generation unit 121 generates a walking waveform can be freely set. For example, the waveform generation unit 121 is configured to continue to generate a walking waveform during a period in which walking of the user is continued. The waveform generation unit 121 may be configured to generate a walking waveform at a specific time point.

Here, a result of verifying a relationship between a value (also referred to as a true value) measured by using motion capture and a value (also referred to as an estimated value) based on sensor data measured by the data acquisition device 11 for detection of an MTC using the detection unit 123 will be described.

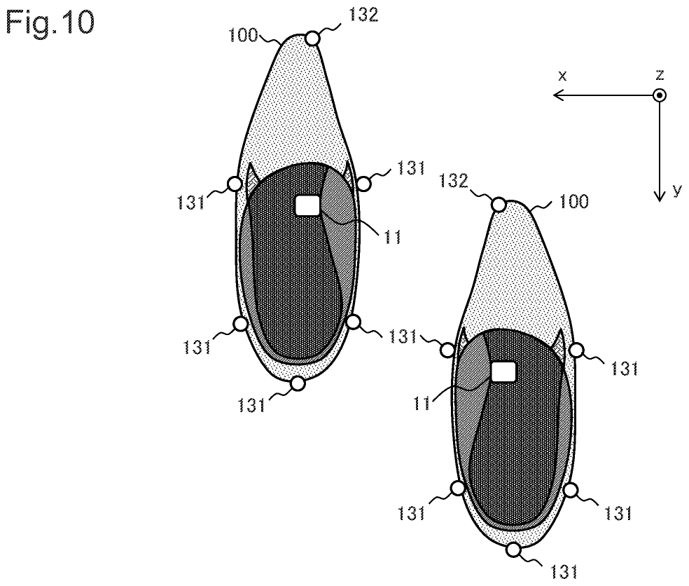

FIG. 10 is a conceptual diagram illustrating an example of measuring a walking parameter by using marks 131 for motion capture and the shoe 100 to which the mark 132 is attached. In the present verification, five marks 131 and one mark 132 were attached to each of the shoes 100 of both feet. The five marks 131 are disposed on the side surface around the opening of the shoe. The five marks 131 are marks for detecting motion of the heel. The center of gravity of a rigid body model that regards the five marks 131 as rigid bodies is detected as a position of the heel. The mark 132 is disposed at a position of the toe of the shoe 100. The mark 132 is used to detect a position of the toe. In the present verification, an intermediate position between the position of the toe and the position of the heel is detected as a midpoint of the foot. The midpoint of the foot may be detected by the mark 131 near the position where the data acquisition device 11 is disposed. The position of the toe, the heel, and the midpoint of the foot are examples of walking parameters. In the present verification, the data acquisition device 11 is installed at a position corresponding to the back side of the arch of each foot.

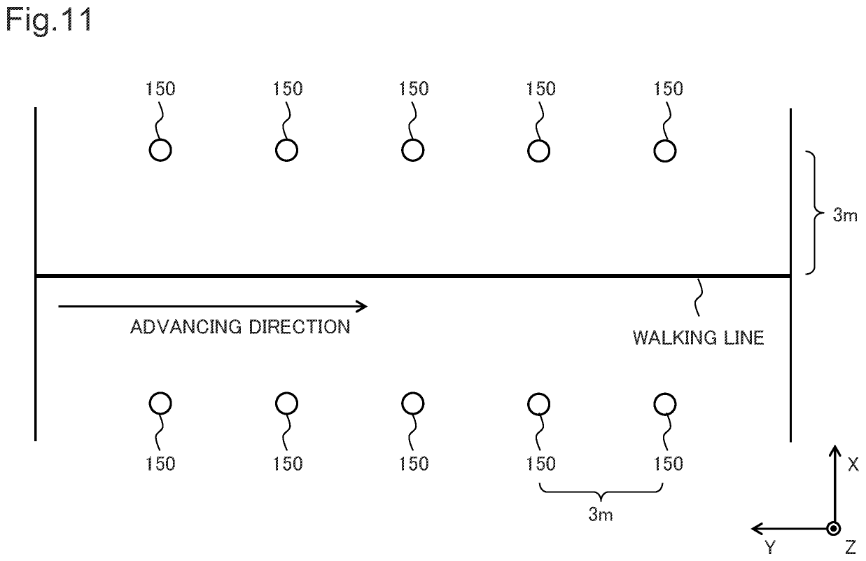

FIG. 11 is a conceptual diagram for describing a walking line and positions at which a plurality of cameras 150 are disposed when a gait of the pedestrian wearing the shoe 100 to which the marks 131 and the mark 132 are attached is verified by using motion capture. In the present verification, five cameras 150 (ten cameras in total) are disposed on each side of the walking line. The plurality of cameras 150 are disposed at an interval of 3 m at a position of 3 m from the walking line. A height of each of the plurality of cameras 150 is fixed at a height of 2 m from a horizontal plane (XY plane). A focal point of each of the plurality of cameras 150 is aligned with the position of the walking line.

Motions of the mark 131 and the mark 132 installed on the shoe 100 of the pedestrian walking along the walking line are analyzed by using moving images captured by the plurality of cameras 150. Motion of the heel is verified by considering the plurality of marks 131 as one rigid body and analyzing the motion of the center of gravity of the marks. Motion of the toe is verified by analyzing the motion of the mark 132. In the present verification, heights of the heel and the toe in the gravity direction (also referred to as vertical heights) and positions of the midpoints of the toe, the heel, and the foot in the advancing direction with respect to the central axis of the body (also referred to as advancing direction positions) are measured by using motion capture.

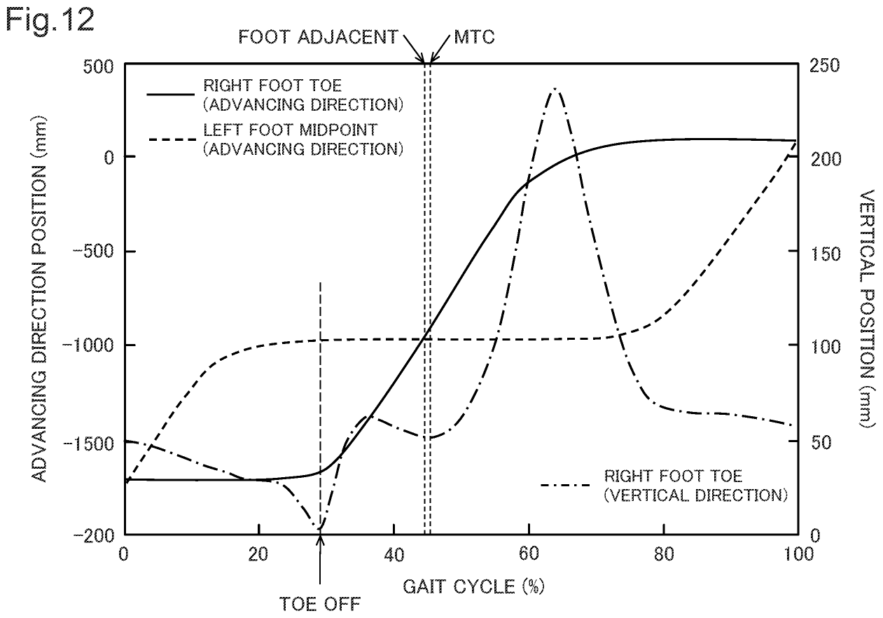

FIG. 12 is a graph illustrating trajectories of positions (advancing direction positions) of the right foot toe and the left foot midpoint in the advancing direction and a trajectory of a position (vertical direction position) of the right foot toe in the vertical direction, which are measured by using motion capture. In FIG. 12, the trajectory of the right foot toe in the advancing direction is indicated by a solid line, the trajectory of the left foot midpoint in the advancing direction is indicated by a dashed line, and the trajectory of the right foot toe in the vertical direction is indicated by a one-dot chain line. In the present example embodiment, a timing at which the right foot toe passes the left foot midpoint in the advancing direction (−Y direction) is defined as a timing of foot adjacent. That is, in FIG. 12, a timing at which the trajectory (solid line) of the right foot toe in the advancing direction intersects the trajectory (dashed line) of the left foot midpoint in the advancing direction corresponds to a timing of foot adjacent. In FIG. 12, a timing at which the trajectory (one-dot chain line) of the right foot toe in the vertical direction becomes minimum around the timing of foot adjacent corresponds to a timing of the MTC. As illustrated in FIG. 12, the timing of foot adjacent and the timing of the MTC are close to each other. In FIG. 12, the timing at which the trajectory (one-dot chain line) of the right foot toe in the vertical direction is minimized corresponds to the timing of toe off.

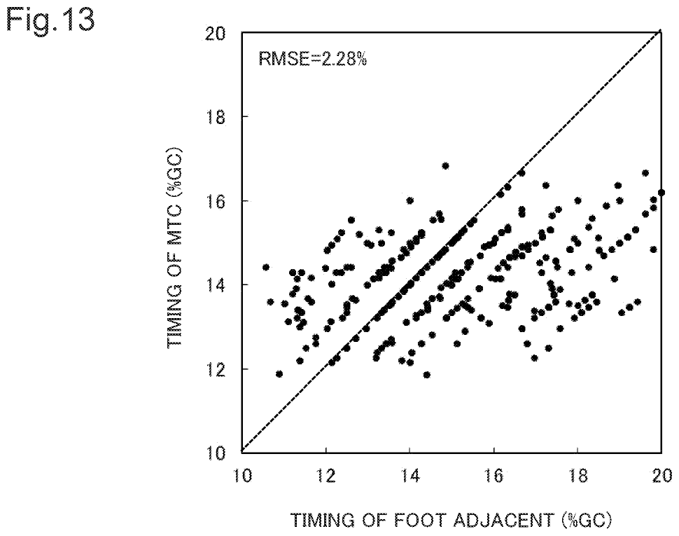

FIG. 13 is a graph illustrating test results for a timing of foot adjacent and a timing of an MTC detected through measurement using motion capture. FIG. 13 relates to walking for a total of 320 steps performed on 26 subjects. In the test results in FIG. 13, a timing of toe off is set as a start point of the gait cycle. In the present verification, the root mean square error (RMSE) regarding a timing of foot adjacent and a timing of the MTC is 2.28 percent (%). That is, the timing of foot adjacent can be regarded as corresponding to the timing of the MTC.

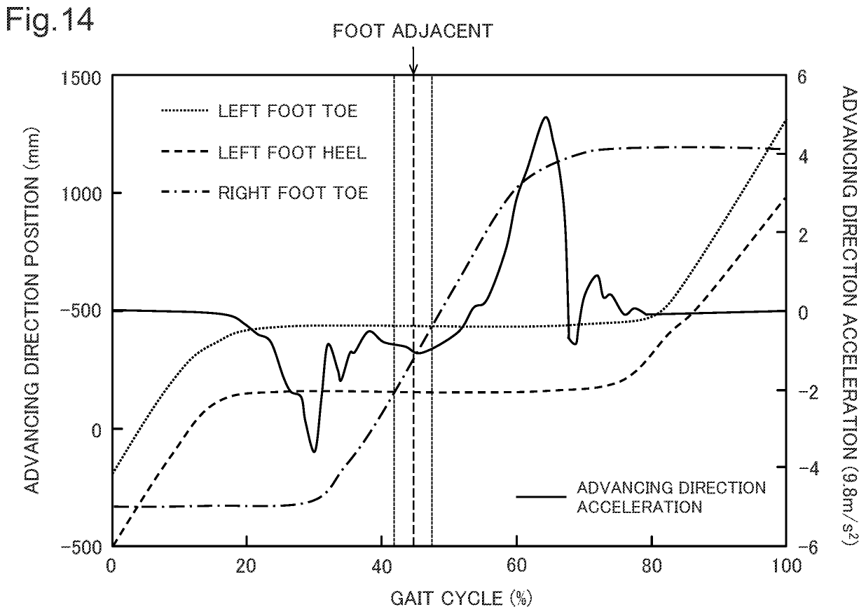

FIG. 14 is a graph illustrating a relationship between a trajectory of an advancing direction position of the foot measured by using motion capture and a walking waveform of an advancing direction acceleration based on sensor data measured by the data acquisition device 11. In FIG. 14, a trajectory of the left foot toe is indicated by a dotted line, a trajectory of the left foot heel is indicated by a dashed line, and a trajectory of the right foot toe is indicated by a one-dot chain line. In FIG. 14, a walking waveform of the advancing direction acceleration is indicated by a solid line. In measurement using motion capture, in the advancing direction, the middle of a timing at which the right foot toe passes the left foot heel and a timing at which the right foot toe passes the left foot toe corresponds to a timing of foot adjacent. At the timing of foot adjacent measured by using motion capture, a gentle downward convex peak is observed in the walking waveform of the advancing direction acceleration. That is, it is estimated that the timing of the gentle downward convex peak appearing between 40 and 60% of the gait cycle starting from the start timing of the support end stage in the walking waveform of the advancing direction acceleration can be used for detection of foot adjacent. In a case where the front in the advancing direction is defined as positive, in the walking waveform of the advancing direction acceleration, the timing of the gentle upward convex peak appearing between 40 and 60% of the gait cycle starting from the start timing of the support end stage corresponds to the timing of foot adjacent. Therefore, in the following description, the timing of the gentle peak appearing between 40 and 60% of the gait cycle starting from the start timing of the support end stage may be expressed as corresponding to the timing of foot adjacent.

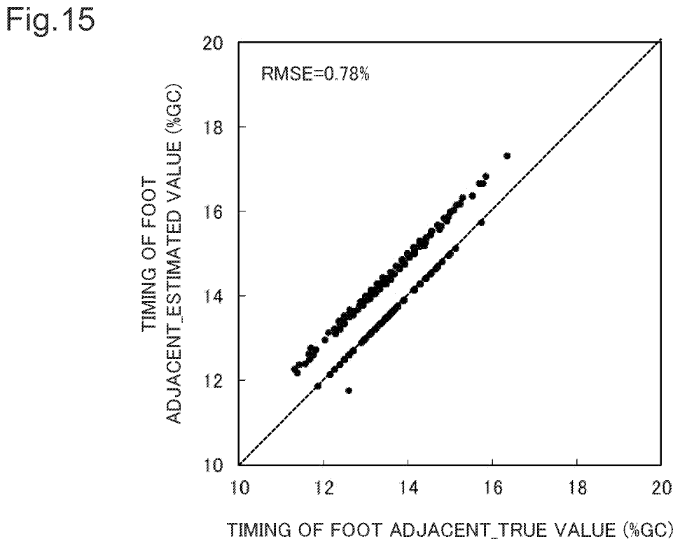

FIG. 15 is a graph illustrating test results for a timing (true value) of foot adjacent measured by using motion capture and a timing (estimated value) of foot adjacent estimated based on the walking waveform of the advancing direction acceleration. FIG. 15 relates to walking for a total of 320 steps performed on 26 subjects. In the test results in FIG. 15, a timing of toe off is set as a start point of the gait cycle. In the present verification, the RMSE regarding the timing (true value) of foot adjacent and the timing (estimated value) of foot adjacent is 0.78%. That is, the timing of the gentle downward convex peak in the walking waveform of the advancing direction acceleration can be used to detect foot adjacent (MTC). In the walking waveform of the advancing direction acceleration, a pattern in which the timing of a gentle peak appearing between 40 and 60% of the gait cycle starting from the start timing of the support end stage is regarded as the timing of the MTC is referred to as a pattern 1.

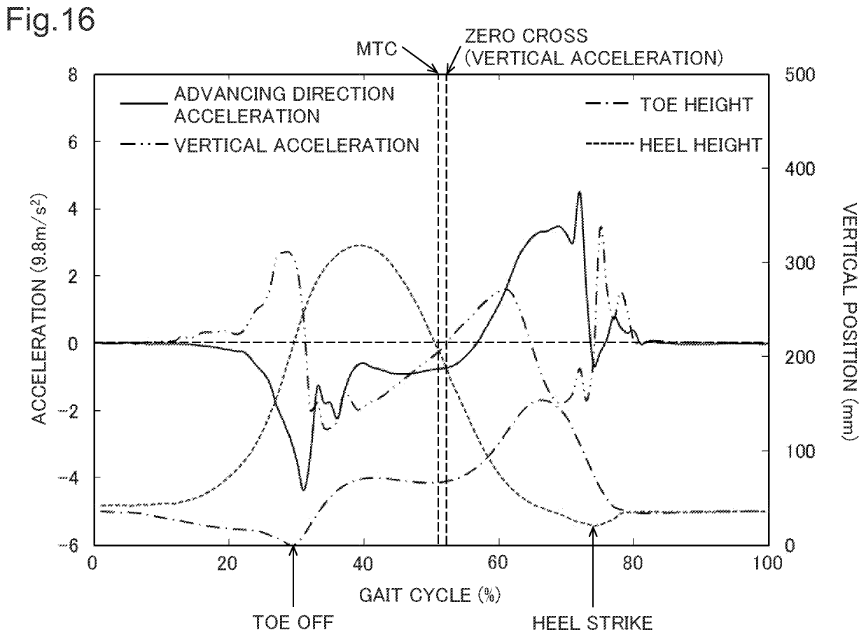

FIG. 16 is a graph illustrating a relationship between a trajectory of a vertical position of the right foot measured by using motion capture and walking waveforms of an advancing direction acceleration and a vertical acceleration based on sensor data measured by the data acquisition device 11. In FIG. 16, regarding a measurement value in motion capture, a trajectory of a toe height is indicated by a one-dot chain line, and a trajectory of a heel height is indicated by a dotted line. In FIG. 16, regarding the sensor data, a walking waveform of an advancing direction acceleration is indicated by a solid line, and a vertical acceleration is indicated by a two-dot chain line. In FIG. 16, a timing at which the trajectory (one-dot chain line) of the toe height is minimized corresponds to a timing of toe off, and a timing at which the trajectory (dotted line) of the heel height is minimized corresponds to a timing of heel strike. In the trajectory (one-dot chain line) of the toe height, the minimum peak between the timing of toe off and the timing of heel strike corresponds to the timing of the MTC. In the vicinity of the timing of the MTC of the trajectory of the toe height (alternate long and short dash line), the walking waveform of the vertical acceleration (two-dot chain line) crosses zero.

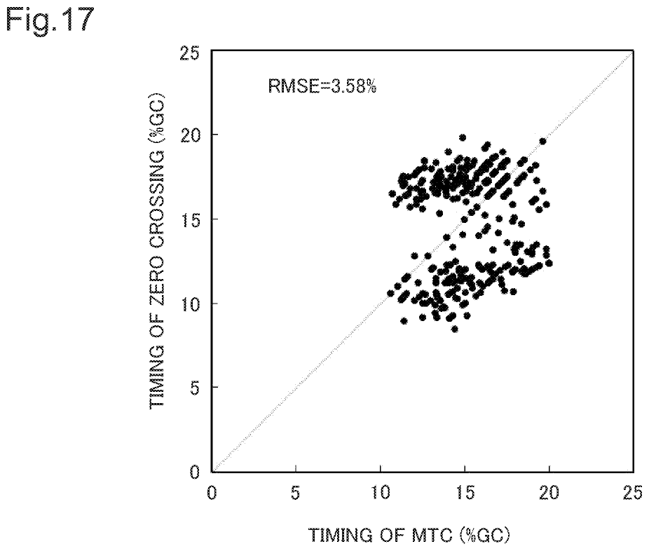

FIG. 17 is a graph illustrating test results for a timing of an MTC in a trajectory of the toe height measured by using motion capture and a timing of zero crossing in a walking waveform of the vertical acceleration. FIG. 17 relates to walking for a total of 320 steps performed on 26 subjects. In the test results in FIG. 17, a timing of toe off is set as a start point of the gait cycle. In the present verification, an RMSE regarding the timing of the MTC in the trajectory of the toe height and the timing of the zero crossing in the walking waveform of the vertical acceleration is 3.58%. That is, the timing of the zero crossing in the walking waveform of the vertical acceleration can be used to detect the MTC. A pattern in which the timing of the zero crossing appearing between 40 to 60% of the gait cycle starting from the start timing of the support end stage in the walking waveform of the vertical acceleration is regarded as the timing of the MTC is referred to as a pattern 2.

The detection unit 123 detects a timing of the MTC from the generated walking waveform. For example, the detection unit 123 detects a timing of foot adjacent from the walking waveform of the advancing direction acceleration (pattern 1). In the pattern 1, the detection unit 123 detects the timing of foot adjacent as the timing of the MTC. For example, the walking index calculation device 12 detects the timing of the zero crossing from the walking waveform of the vertical acceleration (pattern 2). In the pattern 2, the detection unit 123 detects the timing of the zero crossing as the timing of the MTC. The detection unit 123 derives values of the vertical height and the roll angle at the detected timing of the MTC. The value of the vertical height or the value of the roll angle is an example of a walking parameter.

The calculation unit 125 calculates a value of the MTC by using the values of the vertical height and the roll angle at the timing of the MTC. For example, the calculation unit 125 calculates the MTC by applying the values of the vertical height and the roll angle at the timing of the MTC to an algorithm for calculating the MTC. For example, the calculation unit 125 calculates the MTC by applying the values of the vertical height and the roll angle at the timing of the MTC to an MTC estimation model. The estimation model is a model in which the values of the vertical height and the roll angle are used as explanatory variables and the MTC is used as an objective variable. For example, the estimation model is a model generated by supervised learning in which the values of the vertical height and the roll angle are used as explanatory variables and the MTC is used as an objective variable.

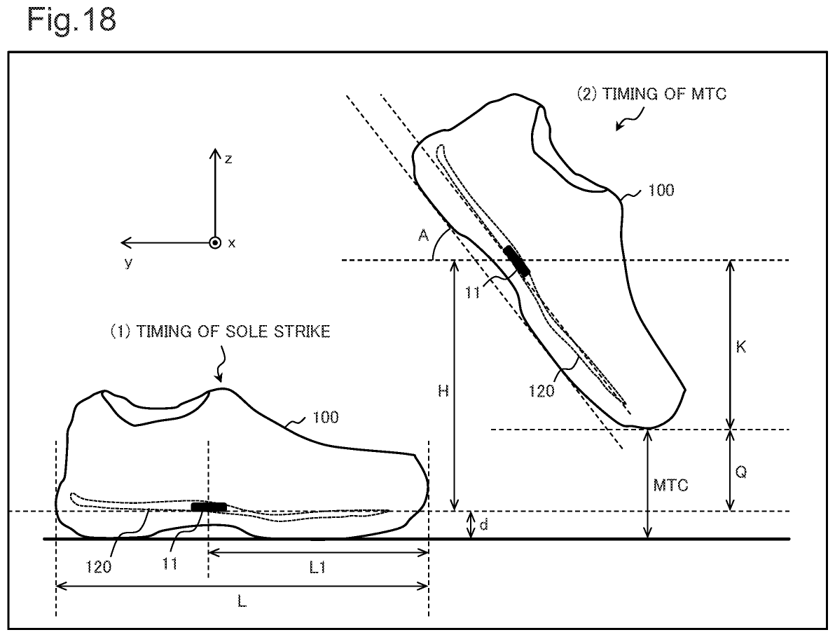

Here, an example of an algorithm for calculating the MTC by using the values of the vertical height and the roll angle at the timing of the MTC will be described. FIG. 18 is a conceptual diagram for describing a method of calculating a value of the MTC. FIG. 18 is a side view of the shoe 100 (right foot) at (1) a timing of sole strike and (2) a timing of the MTC. An insole 120 on which the data acquisition device 11 is mounted is inserted into the shoe 100. The data acquisition device 11 is disposed at a position on the back side of the arch of the foot. A length from the heel to the toe of the shoe 100 is denoted by L. A length from the installation position of the data acquisition device 11 to the toe (also referred to as a sensor position in the advancing direction) is denoted by L1. In the present example embodiment, it is assumed that the sensor position L1 in the advancing direction is known. At the timing of sole strike, a height of the data acquisition device 11 with respect to the ground (also referred to as an initial sensor height) is denoted by d. A difference (vertical height) between the height of the data acquisition device 11 at the timing of the MTC and the height of the data acquisition device 11 at the timing of sole strike is denoted by H. At the timing of the MTC, a distance from the ground to the toe is defined as the MTC. At the timing of the MTC, a vertical height from the height of the data acquisition device 11 to the height of the toe is denoted by K (also referred to as a first value). At the timing of the MTC, a difference between the sensor heights H and K is denoted by Q (also referred to as a second value). The roll angle at the timing of the MTC is denoted by A. In this case, the following Equations 1 to 3 are established.

K=L1×sin A (1)

Q=H−K (2)

MTC=Q+d (3)

In the case of the example in FIG. 18, the calculation unit 125 assigns the value H of the vertical height and the value A of the roll angle at the detected timing of the MTC to the above Equations 1 to 3 to calculate a value of the MTC.

The calculation unit 125 outputs the calculated value of the MTC. For example, the value of the MTC output from the calculation unit 125 is displayed on a screen of a terminal device (not illustrated) carried by the user or a screen of a display device (not illustrated). For example, the value of the MTC output from the calculation unit 125 is output to a system (not illustrated) that analyzes the value of the MTC. For example, the value of the MTC output from the calculation unit 125 is accumulated in a database (not illustrated) and used as big data. A use of the value of the MTC output from the calculation unit 125 is not particularly limited.

<Pattern 1>

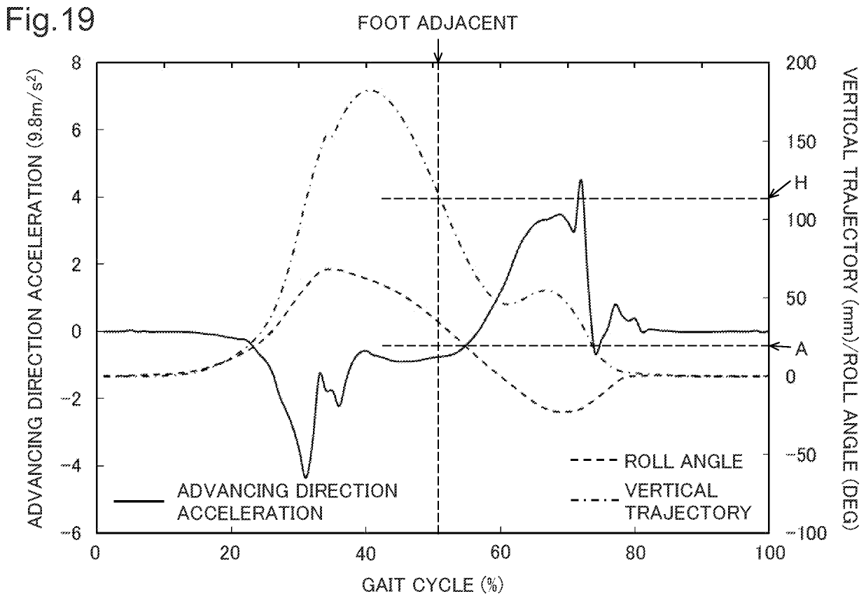

FIG. 19 is a graph for describing calculation of the MTC in the pattern 1. In FIG. 19, a center timing of the support phase (the start of the support end stage) is set as a start point of one gait cycle. In FIG. 19, a walking waveform of the advancing direction acceleration is indicated by a solid line, a walking waveform of the roll angle is indicated by a dashed line, and a walking waveform of the vertical trajectory is indicated by a one-dot chain line.

In the case of the pattern 1, the detection unit 123 detects a timing of foot adjacent from the walking waveform of the advancing direction acceleration. For example, in the walking waveform of the advancing direction acceleration, the detection unit 123 detects the timing of foot adjacent based on a gentle peak appearing in a period of 40 to 60% of the gait cycle starting from the start timing of the support end stage. The timing of foot adjacent is not limited to being based on an extreme value of the gentle peak of the walking waveform of the advancing direction acceleration, and is detected according to a shape of the peak. For example, the detection unit 123 fits a gentle peak of the walking waveform of the advancing direction acceleration to a quadratic curve, and detects a timing of an extreme value of the quadratic curve as the timing of foot adjacent. The calculation unit 125 assigns the value H of the vertical height and the value A of the roll angle at the timing of foot adjacent to the above Equations 1 to 3 to calculate a value of the MTC. The calculation unit 125 may input the value H of the vertical height and the value A of the roll angle at the timing of foot adjacent to an MTC estimation model and estimate an output value therefrom as the MTC.

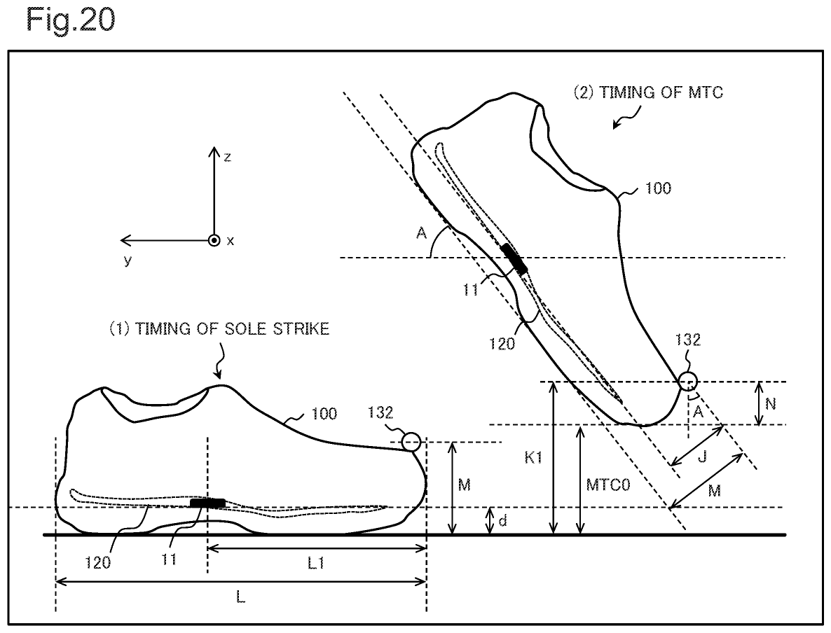

FIG. 20 is a conceptual diagram for describing an example of calculating a true value (MTCO) of the MTC by using motion capture. FIG. 20 is a side view of the shoe 100 (right foot) at (1) a timing of sole strike and (2) a timing of the MTC. The mark 132 in motion capture is installed on the toe of the shoe 100. Measurement using motion capture is performed in the same manner as in the example in FIG. 12. A length from the heel to the toe of the shoe 100 is denoted by L. A length from the installation position of the data acquisition device 11 to the toe (also referred to as a sensor position in the advancing direction) is denoted by L1. In the present example embodiment, it is assumed that the sensor position L1 in the advancing direction is known. At the timing of sole strike, a height of the data acquisition device 11 with respect to the ground (also referred to as an initial sensor height) is denoted by d. At the timing of sole strike, a height of the mark 132 with respect to the ground (also referred to as an initial mark height) is denoted by M. A difference between the initial mark height M and the initial sensor height d is defined by J. A height of the mark 132 with respect to the ground at the timing of the MTC is denoted by K1 (also referred to as a mark height). At the timing of the MTC, a distance from the ground to the toe is denoted by MTCO (true value). At the timing of the MTC, a difference between K1 and MTCO is denoted by N. The roll angle at the timing of the MTC is denoted by A. At this time, the following Equations 4 to 6 are established.

J=M−d (4)

N=J×cos A (5)

MTCO=K1−N (6)

In the case of the example in FIG. 20, if the mark height K1 measured by using motion capture and the value A of the roll angle at the timing of the MTC based on the sensor data acquired by the detection unit 123 are assigned to the above Equations 4 to 6, a value of MTCO is calculated.

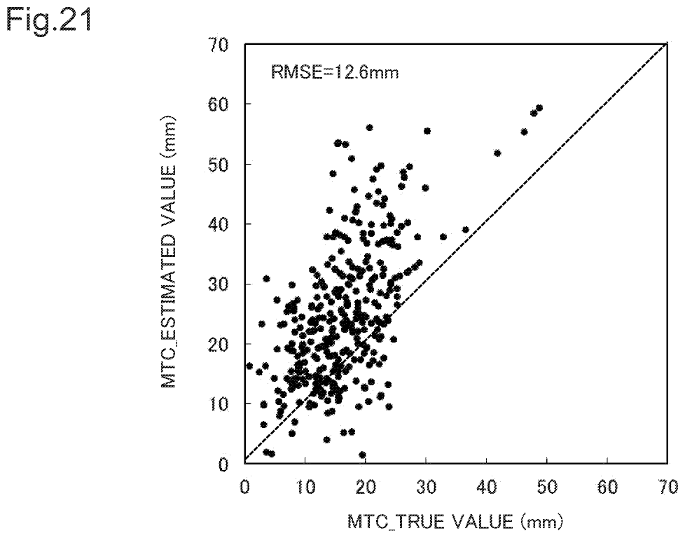

FIG. 21 is a graph illustrating test results for a true value (MTCO) of the MTC measured by using motion capture and an estimated value (MTC) of the MTC calculated based on the timing of foot adjacent detected from the walking waveform of the advancing direction acceleration. FIG. 21 relates to walking for a total of 320 steps performed on 26 subjects. In the present verification, an RMSE for the true value (MTCO) of the MTC and the estimated value (MTC) of the MTC is 12.6 millimeters (mm). An error between the true value of the MTC (MTCO) and the estimated value of the MTC (MTC) is about 10 mm, and can thus be considered to be within an allowable range. That is, the estimated value (MTC) of the MTC calculated based on the pattern 1 can be used for verification of the MTC.

<Pattern 2>

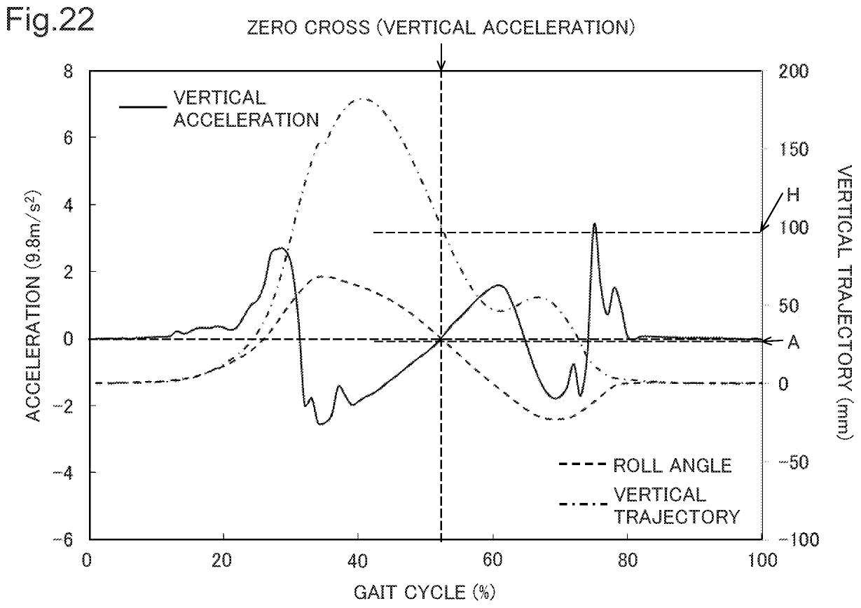

FIG. 22 is a graph for describing calculation of the MTC in the pattern 2. In FIG. 22, a center timing of the support phase (the start of the support end stage) is set as a start point of one gait cycle. In FIG. 22, a walking waveform of the vertical acceleration is indicated by a solid line, a walking waveform of the roll angle is indicated by a dashed line, and a walking waveform of the vertical trajectory is indicated by a one-dot chain line.

In the case of the pattern 2, the detection unit 123 detects a timing of the zero crossing from the walking waveform of the vertical acceleration. For example, in the walking waveform of the vertical acceleration, the detection unit 123 detects a timing of the zero crossing appearing in a period of 40 to 60% of the gait cycle starting from the start timing of the support end stage. The calculation unit 125 assigns the value H of the vertical height and the value A of the roll angle at the timing of the zero crossing to Equations 1 to 3 to calculate a value of the MTC. The calculation unit 125 may input the value H of the vertical height and the value A of the roll angle at the timing of the zero crossing to the MTC estimation model and estimate an output value therefrom as the MTC.

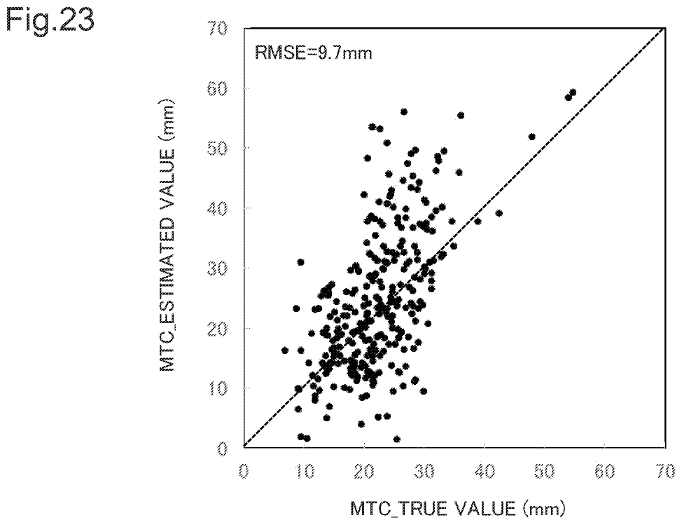

FIG. 23 is a graph illustrating test results for a true value (MTCO) of the MTC measured by using motion capture and an estimated value (MTC) of the MTC calculated based on a timing of the zero crossing detected from the walking waveform of the vertical acceleration. FIG. 23 relates to walking for a total of 320 steps performed on 26 subjects. In the present verification, an RMSE for the true value (MTCO) of the MTC and the estimated value (MTC) of the MTC is 9.7 millimeters (mm). An error between the true value of the MTC (MTCO) and the estimated value of the MTC (MTC) is about 10 mm, and can thus be considered to be within an allowable range. That is, the estimated value (MTC) of the MTC calculated based on the pattern 2 can be used for verification of the MTC.

(Operation)

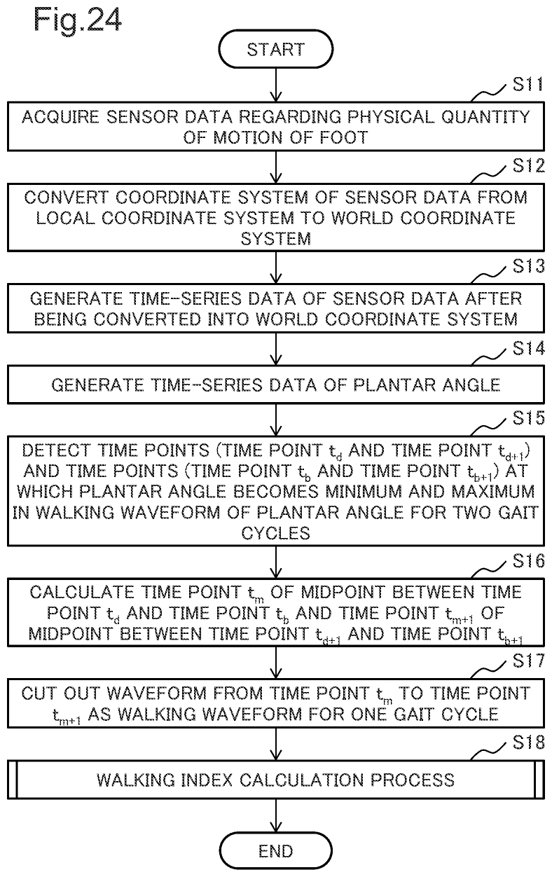

Next, an operation of the walking index calculation device 12 of the walking index calculation system 1 of the present example embodiment will be described with reference to the drawings. FIG. 24 is a flowchart for describing an example of an outline of the operation of the walking index calculation device 12. FIG. 24 relates to a case where a center timing of the support phase (the start of the support end stage) is set as a start point of one gait cycle. In a case where a timing other than the start of the support end stage is set as a start point of one gait cycle, one gait cycle may be cut out in accordance with a timing set as the start point. Hereinafter, the walking index calculation device 12 will be described as an operation subject.

In FIG. 24, first, the walking index calculation device 12 acquires, from the data acquisition device 11, sensor data regarding a physical quantity of motion of a foot of a pedestrian wearing footwear in which the data acquisition device 11 is installed and walking (step S11). The walking index calculation device 12 acquires sensor data in a local coordinate system of the data acquisition device 11. For example, the walking index calculation device 12 acquires a three-dimensional spatial acceleration and a three-dimensional spatial angular velocity from the data acquisition device 11 as sensor data regarding the motion of the foot.

Next, the walking index calculation device 12 converts a coordinate system of the sensor data from the local coordinate system of the data acquisition device 11 to a world coordinate system (step S12).

Next, the walking index calculation device 12 generates time-series data (walking waveform) of the sensor data after conversion into the world coordinate system (step S13).

Next, the walking index calculation device 12 calculates a spatial angle (plantar angle) by using at least one of the spatial acceleration and the spatial angular velocity, and generates time-series data (walking waveform) of a plantar angle (step S14). The walking index calculation device 12 generates time-series data (walking waveform) of a spatial velocity or a spatial trajectory as necessary.

Next, the walking index calculation device 12 detects time points (time point to and time point t_{d+1}) at which the plantar angle becomes minimum and time points (time point t_{b} and time point t_{b+1}) at which the plantar angle becomes maximum in the walking waveform (walking waveform) of the plantar angle for two gait cycles (step S15).

Next, the walking index calculation device 12 calculates a time point tₘ of the midpoint between the time point t_{d} and the time point t_{b} and a time point tₘ₊₁ of the midpoint between the time point t_{d+1} and the time point t_{b+1} (step S16).

Next, the walking index calculation device 12 cuts out a waveform from the time point tₘ to the time point tₘ₊₁ as a walking waveform for one gait cycle (step S17).

The walking index calculation device 12 executes a walking index calculation process by using the cutout walking waveform for one gait cycle (step S18). The walking index calculation process will be described later.

[Walking Index Calculation Process]

Next, an outline of the walking index calculation process in step S18 in FIG. 24 will be described with reference to the drawings. Hereinafter, the walking index calculation process will be described separately for the pattern 1 and the pattern 2. In the following description of the walking index calculation process, the walking index calculation device 12 will be described as an operation subject.

<Pattern 1>

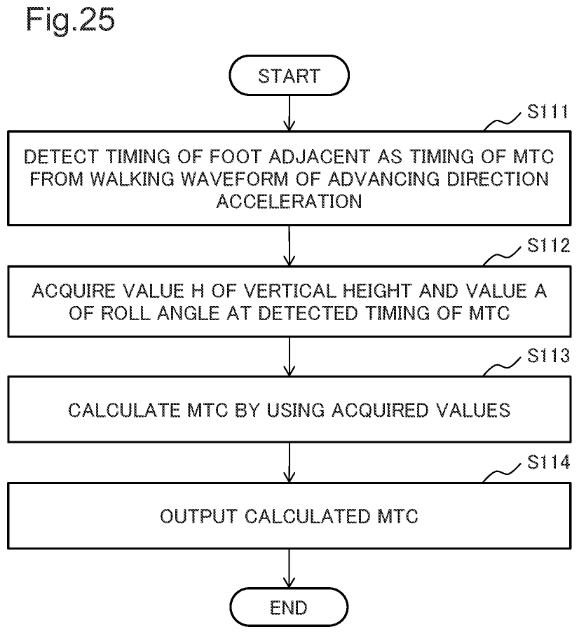

FIG. 25 is a flowchart for describing an example of a walking index calculation process in the pattern 1. In FIG. 25, first, the walking index calculation device 12 detects a timing of foot adjacent as a timing of the MTC from the walking waveform of the advancing direction acceleration (step S111).

Next, the walking index calculation device 12 acquires the value H of the vertical height and the value A of the roll angle at the detected timing of the MTC (step S112). The walking index calculation device 12 acquires the value H of the vertical height from the walking waveform of the vertical trajectory, and acquires the value A of the roll angle from the walking waveform of the roll angle.

Next, the walking index calculation device 12 calculates the MTC by using the acquired value (step S113). For example, the walking index calculation device 12 calculates the value of the MTC by applying the value H of the vertical height and the value A of the roll angle at the timing of the MTC to Equations 1 to 3.

Next, the walking index calculation device 12 outputs the calculated MTC (step S114). For example, the value of the MTC output from the walking index calculation device 12 is displayed on a screen of a terminal device (not illustrated) carried by the user or a screen of a display device (not illustrated). For example, the value of the MTC output from the walking index calculation device 12 is output to a system (not illustrated) that analyzes the value of the MTC. For example, the value of the MTC output from the walking index calculation device 12 is accumulated in a database (not illustrated) and used as big data.

<Pattern 2>

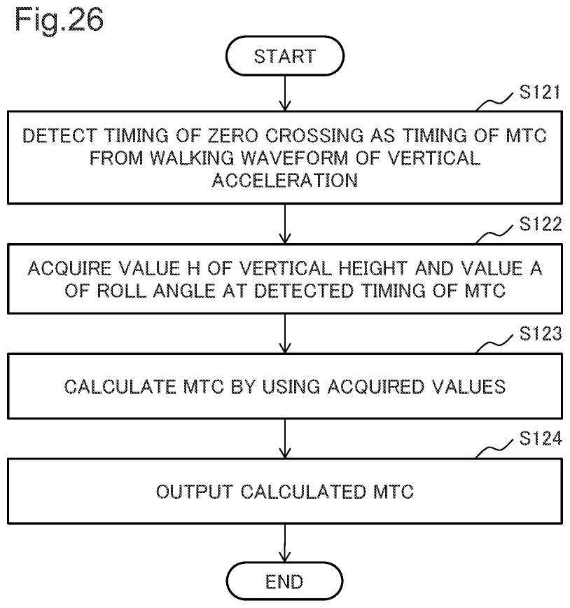

FIG. 26 is a flowchart for describing an example of a walking index calculation process in the pattern 2. In FIG. 26, first, the walking index calculation device 12 detects a timing of the zero crossing as the timing of the MTC from the walking waveform of the vertical acceleration (step S121).

Next, the walking index calculation device 12 acquires the value H of the vertical height and the value A of the roll angle at the timing of the detected the MTC (step S122). The walking index calculation device 12 acquires the value H of the vertical height from the walking waveform of the vertical trajectory, and acquires the value A of the roll angle from the walking waveform of the roll angle.

Next, the walking index calculation device 12 calculates the MTC by using the acquired value (step S123). For example, the walking index calculation device 12 calculates the value of the MTC by applying the value H of the vertical height and the value A of the roll angle at the timing of the MTC to Equations 1 to 3.

Next, the walking index calculation device 12 outputs the calculated MTC (step S124). For example, the value of the MTC output from the walking index calculation device 12 is displayed on a screen of a terminal device (not illustrated) carried by the user or a screen of a display device (not illustrated). For example, the value of the MTC output from the walking index calculation device 12 is output to a system (not illustrated) that analyzes the value of the MTC. For example, the value of the MTC output from the walking index calculation device 12 is accumulated in a database (not illustrated) and used as big data.

As described above, the walking index calculation system of the present example embodiment includes the data acquisition device and the walking index calculation device. The data acquisition device is disposed on footwear worn by a user who is a measurement target of a walking waveform. The data acquisition device measures a spatial acceleration and a spatial angular velocity according to walking of the user, and generates sensor data based on the measured spatial acceleration and spatial angular velocity. The data acquisition device transmits the generated sensor data to the walking index calculation device. The walking index calculation device includes a waveform generation unit, a detection unit, and a calculation unit. The waveform generation unit generates a walking waveform by using the sensor data regarding motion of the foot acquired by the sensor installed in the footwear. The detection unit detects a timing at which the clearance of the toe is minimized from the walking waveform. The calculation unit calculates the minimum value of the clearance of the toe by using a walking parameter at the timing at which the clearance of the toe is minimized.

In the present example embodiment, the data acquisition device may be installed on footwear of a user who lives a daily life. The walking index calculation device calculates the minimum value of the clearance of the toe in walking of the user who lives a daily life by using the sensor data acquired by the data acquisition device. For example, the walking index calculation device can calculate the clearance of the toe in walking of the user by using walking waveforms of the plantar angle, the advancing direction acceleration, the vertical trajectory, and the roll angle among the walking waveforms that are time-series data of the sensor data. For example, the walking index device can calculate the clearance of the toe in walking of the user by using the walking waveforms of the plantar angle, the vertical acceleration, the vertical trajectory, and the roll angle among the walking waveforms that are the time-series data of the sensor data. That is, according to the present example embodiment, the clearance of the toe can be calculated without calculating all the trajectories of the foot, the heel, and the toe. Therefore, according to the present example embodiment, since the load of calculation can be reduced, the clearance of the toe can be calculated in walking in daily life.

In one aspect of the present example embodiment, the calculation unit calculates the minimum value of the clearance of the toe by using a value of a height of the sensor detected from the walking waveform of the vertical trajectory and a value of a rotation angle in the sagittal plane detected from a walking waveform of the rotation angle in the sagittal plane at the timing of the MTC. According to the present aspect, the minimum value of the clearance of the toe can be calculated by using the walking parameter detected from the walking waveform.

In one aspect of the present example embodiment, in the walking waveform of the advancing direction acceleration, the detection unit detects a timing of a gentle peak appearing between 40 and 60% of the gait cycle starting from the start timing of the support end stage as a timing at which the clearance of the toe is minimized. According to the present aspect, the minimum value of the clearance of the toe can be calculated by detecting a timing of foot adjacent detected from the walking waveform of the advancing direction acceleration as a timing at which the clearance of the toe is minimized.

In one aspect of the present example embodiment, the detection unit detects, as a timing at which the clearance of the toe is minimized, a timing of the zero crossing appearing between 40 and 60% of the gait cycle starting from the start timing of the support end stage in the walking waveform of the vertical acceleration. According to the present aspect, the minimum value of the clearance of the toe can be calculated by detecting a timing of the zero crossing detected from the walking waveform of the vertical acceleration as a timing at which the clearance of the toe is minimized.

In one aspect of the present example embodiment, the calculation unit calculates a first value by multiplying a sine of the rotation angle in the sagittal plane at the timing at which the clearance of the toe is minimized by a position of the sensor in the advancing direction. The calculation unit calculates a second value by subtracting the first value from the height of the sensor at the timing at which the clearance of the toe is minimized. The calculation unit adds the value of the height of the sensor at the timing of sole strike to the second value to calculate the minimum value of the clearance of the toe. According to the present aspect, the minimum value of the clearance of the toe can be calculated by using a walking parameter acquired from the walking waveform.

### Second Example Embodiment

Next, a walking index calculation system according to a second example embodiment will be described with reference to the drawings. The walking index calculation system of the present example embodiment is different from that of the first example embodiment in that a length from an installation position of a data acquisition device to a toe (also referred to as a sensor position in an advancing direction) is calculated.

(Configuration)

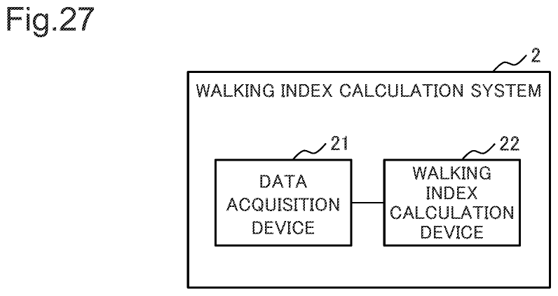

FIG. 27 is a block diagram illustrating a configuration of a walking index calculation system 2 of the present example embodiment. The walking index calculation system 2 includes a data acquisition device 21 and a walking index calculation device 22. The data acquisition device 21 and the walking index calculation device 22 may be connected by wire or wirelessly. The data acquisition device 21 and the walking index calculation device 22 may be configured by a single device. The walking index calculation system 2 may include only the walking index calculation device 22 without including the data acquisition device 21. Since the data acquisition device 21 has the same configuration as the data acquisition device 11 of the first example embodiment, a detailed description thereof will be omitted.

[Walking Index Calculation Device]

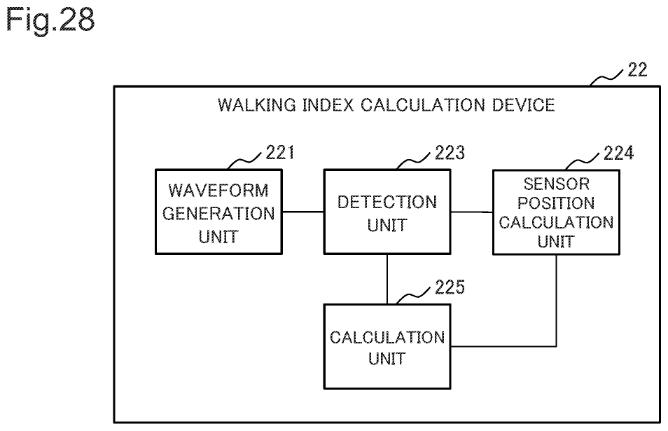

Next, details of the walking index calculation device 22 will be described with reference to the drawings. FIG. 28 is a block diagram illustrating an example of a configuration of the walking index calculation device 22. The walking index calculation device 22 includes a waveform generation unit 221, a detection unit 223, a sensor position calculation unit 224, and a calculation unit 225.

The waveform generation unit 221 acquires sensor data from the data acquisition device 21 (sensor) installed in footwear worn by a pedestrian. By using the sensor data, the waveform generation unit 221 generates time-series data (also referred to as a walking waveform) associated with walking of the pedestrian wearing the footwear in which the data acquisition device 21 is installed. Since the waveform generation unit 221 has the same configuration as that of the waveform generation unit 121 of the first example embodiment, a detailed description thereof will be omitted.

The detection unit 223 detects a walking event from the walking waveform generated by the waveform generation unit 221. The detection unit 223 acquires a value of a walking parameter at a timing of the detected walking event.

For example, the detection unit 223 detects a timing of foot adjacent from a walking waveform of an advancing direction acceleration (pattern 1). In the pattern 1, the timing of foot adjacent detected from the walking waveform of the advancing direction acceleration corresponds to a timing of an MTC. For example, the detection unit 223 detects the timing of the zero crossing from the walking waveform of the vertical acceleration (pattern 2). In the pattern 2, the timing of the zero crossing detected from the walking waveform of the vertical acceleration corresponds to the timing of the MTC. The detection unit 223 acquires a vertical height and a roll angle at the detected the timing of the MTC. The detection unit 223 acquires a value of the vertical height at the timing of the MTC from a walking waveform of a vertical trajectory. The detection unit 223 acquires a value of the vertical height at the timing of the MTC from a walking waveform of the roll angle.

The sensor position calculation unit 224 detects the timing of toe off from the walking waveform generated by the waveform generation unit 221. For example, the sensor position calculation unit 224 detects a timing of toe off from the walking waveform of the advancing direction acceleration. The sensor position calculation unit 224 acquires a value of the vertical height at the timing of toe off from the walking waveform of the vertical trajectory. The sensor position calculation unit 224 acquires a value of the roll angle at the timing of toe off from the walking waveform of the roll angle. The sensor position calculation unit 224 calculates a sensor position in the advancing direction by using the value of the vertical height and the value of the roll angle at the timing of toe off.

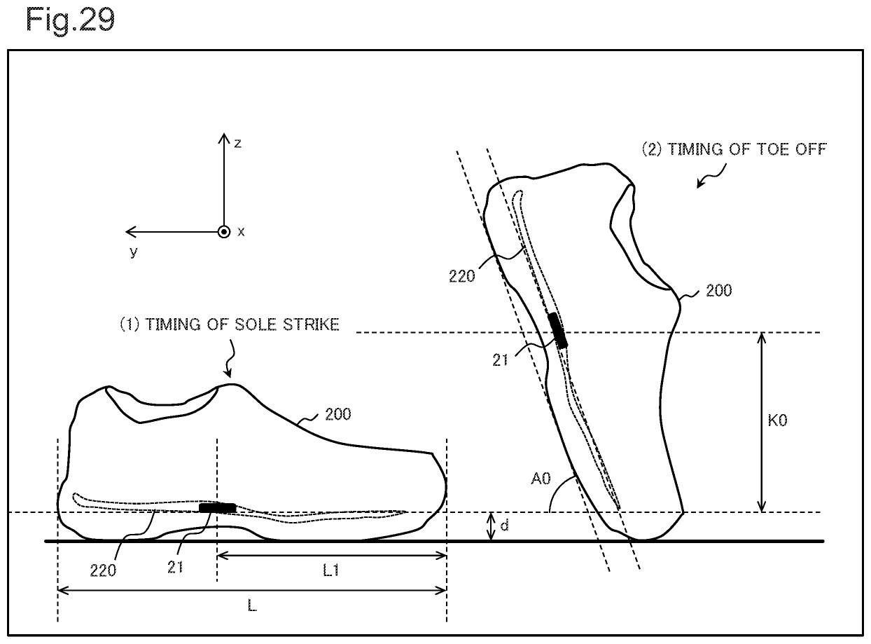

FIG. 29 is a conceptual diagram for describing a method of calculating a sensor position L1 in the advancing direction. FIG. 29 is a side view of a shoe 200 at (1) a timing of sole strike and (2) a timing of toe off. An insole 220 on which the data acquisition device 21 is mounted is inserted into the shoe 200. The data acquisition device 21 is disposed at a position on the back side of the arch of the foot. A length from the heel to the toe of the shoe 200 is denoted by L. In the present example embodiment, it is assumed that the sensor position L1 in the advancing direction is unknown. At the timing of sole strike, a height of the data acquisition device 21 with respect to the ground (also referred to as an initial sensor height) is denoted by d. A difference (vertical height) between the height of the data acquisition device 21 at the timing of toe off and the height of the data acquisition device 21 at the timing of sole strike is denoted by K0. The roll angle at the timing of toe off is A0. In this case, the following Equation 7 is established.

L1=(K0+d)/sin A0 (7).

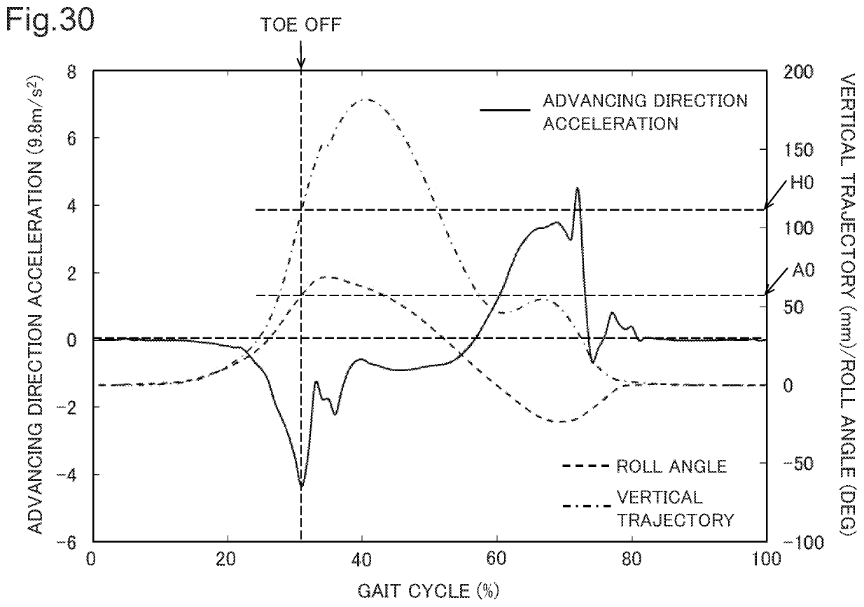

FIG. 30 is a graph for describing calculation of the sensor position L1 in the advancing direction at the timing of toe off. In FIG. 30, a center timing of the support phase (the start of the support end stage) is set as a start point of one gait cycle. In FIG. 30, a walking waveform of the advancing direction acceleration is indicated by a solid line, a walking waveform of the roll angle is indicated by a dashed line, and a walking waveform of the vertical trajectory is indicated by a one-dot chain line. The detection unit 223 detects a timing of toe off from the walking waveform of the advancing direction acceleration. For example, the detection unit 223 detects the timing of toe off based on a peak of the walking waveform of the advancing direction acceleration appearing in a period of 20 to 40% of the gait cycle starting from the start timing of the support end stage. The timing of toe off corresponds to a timing at which the advancing direction acceleration becomes an extreme value. The sensor position calculation unit 224 assigns a value H0 of the vertical height and a value A0 of the roll angle at the detected timing of toe off to the above Equation 7, and calculates a value of the sensor position L1 in the advancing direction.

The calculation unit 225 calculates a value of the MTC by using the values of the vertical height and the roll angle at the timing of the MTC. For example, the calculation unit 225 calculates the value of the MTC by using the value of the sensor position L1 in the advancing direction calculated by the sensor position calculation unit 224 and the values of the vertical height and the roll angle at the timing of the MTC. For example, the calculation unit 225 calculates the value of the MTC by applying the value of the sensor position L1, the value of the vertical height, and the value of the roll angle to an algorithm for calculating the MTC. A method of calculating the value of the MTC by using the calculation unit 225 is similar to that in the first example embodiment.

The calculation unit 225 outputs the calculated the value of the MTC. For example, the value of the MTC output from the calculation unit 225 is displayed on a screen of a terminal device (not illustrated) carried by a user or a screen of a display device (not illustrated). For example, the value of the MTC output from the calculation unit 225 is output to a system (not illustrated) that analyzes the value of the MTC. For example, the value of the MTC output from the calculation unit 225 is accumulated in a database (not illustrated) and used as big data. The use of the value of the MTC output from the calculation unit 225 is not particularly limited.

(Operation)

Next, an operation of the walking index calculation device 22 of the walking index calculation system 2 of the present example embodiment will be described with reference to the drawings. Since an outline of the operation of the walking index calculation device 22 is similar to that of the first example embodiment (FIG. 24), a description thereof will be omitted. Hereinafter, a walking index calculation process (step S18 in FIG. 24) performed by the walking index calculation device 22 will be described.

[Walking Index Calculation Process]

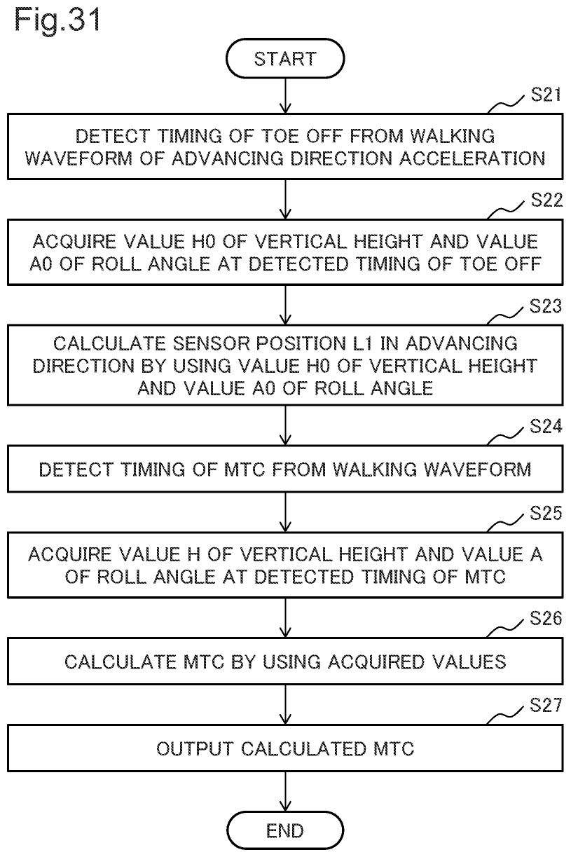

FIG. 31 is a flowchart for describing an example of the walking index calculation process. In FIG. 31, first, the walking index calculation device 22 detects a timing of toe off from the walking waveform of the advancing direction acceleration (step S21).

Next, the walking index calculation device 22 acquires the value H0 of the vertical height and the value A0 of the roll angle at the detected timing of toe off (step S22). The walking index calculation device 22 acquires the value H0 of the vertical height at the timing of toe off from the walking waveform of the vertical acceleration. The walking index calculation device 22 acquires the value A0 of the roll angle at the timing of toe off from the walking waveform of the roll angle.

Next, the walking index calculation device 22 calculates the sensor position L1 in the advancing direction by using the value H0 of the vertical height and the value A0 of the roll angle at the timing of toe off (step S23).

Next, the walking index calculation device 22 detects a timing of the MTC from the walking waveform (step S24). For example, the walking index calculation device 22 detects the timing of the MTC from the walking waveform based on the pattern 1 or the pattern 2.

Next, the walking index calculation device 22 acquires the value H of the vertical height and the value A of the roll angle at the detected timing of the MTC (step S25). The walking index calculation device 22 acquires the value H of the vertical height from the walking waveform of the vertical trajectory, and acquires the value A of the roll angle from the walking waveform of the roll angle.

Next, the walking index calculation device 22 calculates the MTC by using the acquired value (step S26). For example, the walking index calculation device 22 calculates the value of the MTC by using the sensor position L1 in the advancing direction, and the value H of the vertical height and the value A of the roll angle at the timing of the MTC.

Next, the walking index calculation device 22 outputs the calculated MTC (step S27). For example, the value of the MTC output from the walking index calculation device 22 is displayed on a screen of a terminal device (not illustrated) carried by the user or a screen of a display device (not illustrated). For example, the value of the MTC output from the walking index calculation device 22 is output to a system (not illustrated) that analyzes the value of the MTC. For example, the value of the MTC output from the walking index calculation device 22 is accumulated in a database (not illustrated) and used as big data.

As described above, the walking index calculation system of the present example embodiment includes the data acquisition device and the walking index calculation device. The data acquisition device is disposed on footwear worn by a user who is a measurement target of a walking waveform. The data acquisition device measures a spatial acceleration and a spatial angular velocity according to walking of the user, and generates sensor data based on the measured spatial acceleration and spatial angular velocity. The data acquisition device transmits the generated sensor data to the walking index calculation device. The walking index calculation device includes a waveform generation unit, a detection unit, a sensor position calculation unit, and a calculation unit. The waveform generation unit generates a walking waveform by using the sensor data regarding motion of the foot acquired by the sensor installed in the footwear. The detection unit detects a timing at which the clearance of the toe is minimized from the walking waveform. The sensor position calculation unit calculates a position of the sensor in the advancing direction by using a walking parameter at the timing of toe off detected from the walking waveform. The calculation unit calculates the minimum value of the clearance of the toe by using the position of the sensor in the advancing direction calculated by the sensor position calculation unit and the walking parameter at the timing at which the clearance of the toe is minimized.

In the present example embodiment, the position of the sensor in the advancing direction is calculated by using the walking parameter at the timing of toe off. Therefore, even in a case where the position of the sensor in the advancing direction is unknown or the position of the sensor in the advancing direction varies, the clearance of the toe can be calculated.

### Third Example Embodiment

Next, a walking index calculation system according to a third example embodiment will be described with reference to the drawings. A walking index calculation system of the present example embodiment is different from that of the first and second example embodiments in that a calculated value of an MTC is verified.

(Configuration)

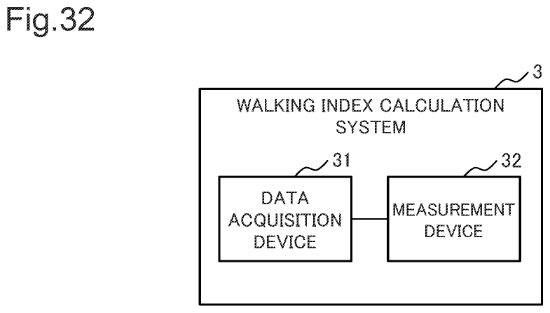

FIG. 32 is a block diagram illustrating a configuration of a walking index calculation system 3 of the present example embodiment. The walking index calculation system 3 includes a data acquisition device 31 and a walking index calculation device 32. The data acquisition device 31 and the walking index calculation device 32 may be connected by wire or wirelessly. The data acquisition device 31 and the walking index calculation device 32 may be configured by a single device. The walking index calculation system 3 may include only the walking index calculation device 32 without including the data acquisition device 31. Since the data acquisition device 31 has the same configuration as that of the data acquisition device 11 of the first example embodiment, a detailed description thereof will be omitted.

[Walking Index Calculation Device]

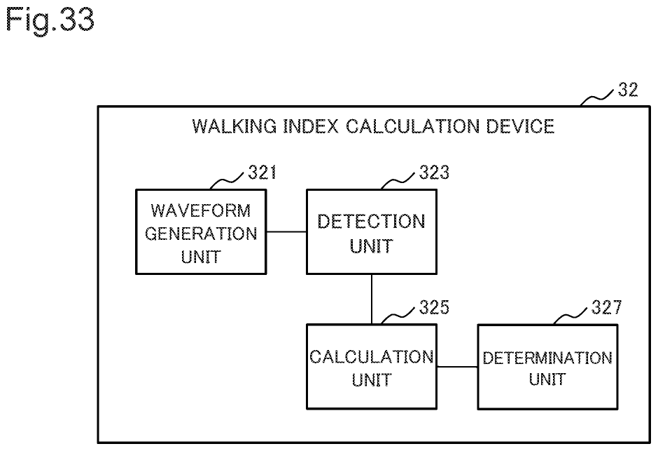

Next, details of the walking index calculation device 32 will be described with reference to the drawings. FIG. 33 is a block diagram illustrating an example of a configuration of the walking index calculation device 32. The walking index calculation device 32 includes a waveform generation unit 321, a detection unit 323, a calculation unit 325, and a determination unit 327.

The waveform generation unit 321 acquires sensor data from the data acquisition device 31 (sensor) installed in footwear worn by a pedestrian. By using the sensor data, the waveform generation unit 321 generates time-series data (also referred to as a walking waveform) associated with walking of the pedestrian wearing the footwear in which the data acquisition device 31 is installed. Since the waveform generation unit 321 has the same configuration as that of the waveform generation unit 121 of the first example embodiment, a detailed description thereof will be omitted.

The detection unit 323 detects a walking event from a walking waveform generated by the waveform generation unit 321. The detection unit 323 acquires a value of a walking parameter at a timing of the detected walking event.

For example, the detection unit 323 detects a timing of foot adjacent from a walking waveform of an advancing direction acceleration (pattern 1). In the pattern 1, the timing of foot adjacent detected from the walking waveform of the advancing direction acceleration corresponds to a timing of an MTC. For example, the detection unit 323 detects the timing of the zero crossing from the walking waveform of the vertical acceleration (pattern 2). In the pattern 2, the timing of the zero crossing detected from the walking waveform of the vertical acceleration corresponds to the timing of the MTC. The detection unit 323 acquires a vertical height and a roll angle at the detected the timing of the MTC. The detection unit 323 acquires a value of the vertical height at the timing of the MTC from a walking waveform of a vertical trajectory. The detection unit 323 acquires a value of the vertical height at the timing of the MTC from a walking waveform of the roll angle.

The calculation unit 325 calculates a value of the MTC by using the values of the vertical height and the roll angle at the timing of the MTC. For example, the calculation unit 325 calculates the value of the MTC by applying the values of the vertical height and the roll angle at the timing of the MTC to an algorithm for calculating the MTC. A method of calculating the value of the MTC by using the calculation unit 325 is similar to that in the first example embodiment.

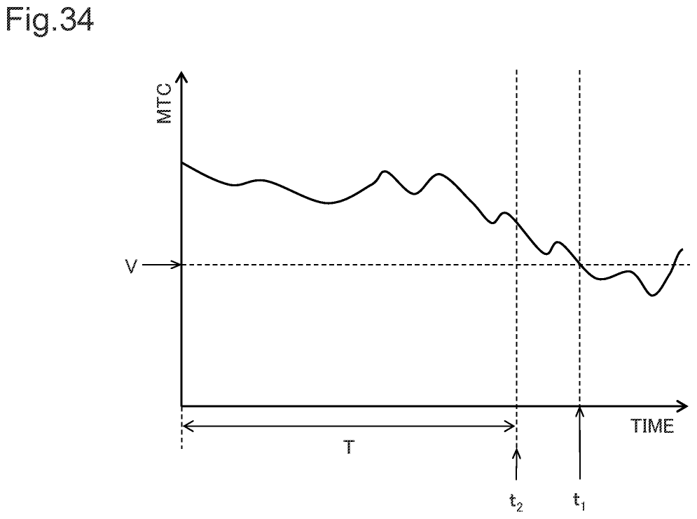

The determination unit 327 verifies the value of the MTC calculated by the calculation unit 325. The determination unit 327 outputs a determination result based on the value of the MTC. FIG. 34 is a graph for describing an example of determination for the value of the MTC using the determination unit 327. For example, the determination unit 327 determines that the risk of falling has occurred at a timing point t₁ at which the value of the MTC is smaller than a threshold value V. For example, the determination unit 327 determines that the risk of falling has occurred at a timing point t₂ at which a certain period T has elapsed since the value of the MTC showed a decreasing tendency.

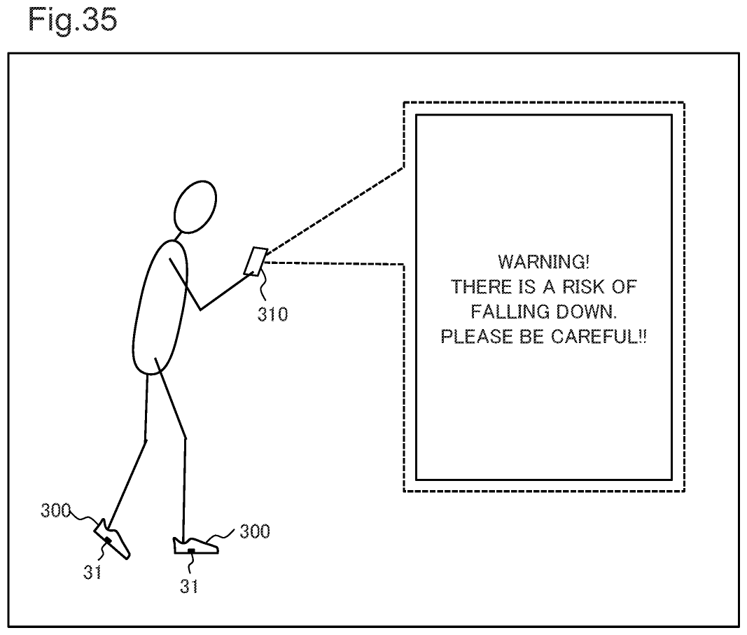

FIG. 35 is a conceptual diagram illustrating an example of displaying a determination result output from the determination unit 327. In the example in FIG. 35, it is assumed that an application having the function of the walking index calculation device 32 is installed in a mobile terminal 310 carried by a user wearing shoes 300 in which the data acquisition device 31 is mounted and walking. For example, on a screen of the mobile terminal 310, a determination result from the determination unit 327 that “Warning! There is a risk of falling down. Please be careful!!” is displayed. For example, the mobile terminal 310 emits a notification sound in accordance with a timing at which the determination result from the determination unit 327 is displayed on the screen of the mobile terminal 310. For example, the mobile terminal 310 is vibrated at the timing at which the determination result from the determination unit 327 is displayed on the screen of the mobile terminal 310. With this configuration, it is possible to notify the user that the determination result is displayed on the screen of the mobile terminal 310. A pedestrian walking while carrying the mobile terminal 310 can perceive that a notification has been issued for his/her walking by a notification sound emitted from the mobile terminal 310 or vibration of the mobile terminal 310. The pedestrian walking while carrying the mobile terminal 310 can recognize notification details in his/her walking by visually recognizing the determination result displayed on the screen of the mobile terminal 310.

(Operation)

Next, an operation of the walking index calculation device 32 of the walking index calculation system 3 of the present example embodiment will be described with reference to the drawings. Since an outline of the operation of the walking index calculation device 32 is similar to that of the first example embodiment (FIG. 24), a description thereof will be omitted. Hereinafter, the walking index calculation process (step S18 in FIG. 24) performed by the walking index calculation device 32 will be described.

[Walking Index Calculation Process]

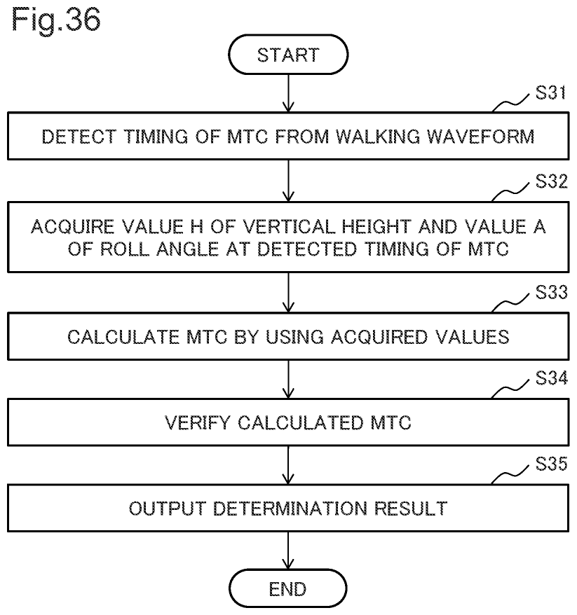

FIG. 36 is a flowchart for describing an example of the walking index calculation process. In FIG. 36, first, the walking index calculation device 32 detects a timing of the MTC from the walking waveform (step S31). For example, the walking index calculation device 32 detects the timing of the MTC from the walking waveform based on the pattern 1 or the pattern 2.

Next, the walking index calculation device 32 acquires the value H of the vertical height and the value A of the roll angle at the detected timing of the MTC (step S32). The walking index calculation device 32 acquires the value H of the vertical height from the walking waveform of the vertical trajectory, and acquires the value A of the roll angle from the walking waveform of the roll angle.

Next, the walking index calculation device 32 calculates the MTC by using the acquired values (step S33). For example, the walking index calculation device 32 calculates a value of the MTC by using the value H of the vertical height and the value A of the roll angle at the timing of the MTC.

Next, the walking index calculation device 32 verifies the calculated MTC (step S34). For example, the walking index calculation device 32 determines that the risk of falling has occurred at a timing point t₁ at which the value of the MTC is smaller than a threshold value V. For example, the walking index calculation device 32 determines that the risk of falling has occurred at a timing point t₂ at which a certain period T has elapsed since the value of the MTC showed a decreasing tendency.

Next, the walking index calculation device 32 outputs a determination result (step S35). For example, the determination result output from the walking index calculation device 32 is displayed on the screen of the mobile terminal 310 carried by the user.

As described above, the walking index calculation system of the present example embodiment includes the data acquisition device and the walking index calculation device. The data acquisition device is disposed on footwear worn by a user who is a measurement target of a walking waveform. The data acquisition device measures a spatial acceleration and a spatial angular velocity according to walking of the user, and generates sensor data based on the measured spatial acceleration and spatial angular velocity. The data acquisition device transmits the generated sensor data to the walking index calculation device. The walking index calculation device includes a waveform generation unit, a detection unit, a calculation unit, and a determination unit. The waveform generation unit generates a walking waveform by using the sensor data regarding motion of the foot acquired by the sensor installed in the footwear. The detection unit detects a timing at which the clearance of the toe is minimized from the walking waveform. The sensor position calculation unit calculates a position of the sensor in the advancing direction by using a walking parameter at the timing of toe off detected from the walking waveform. The calculation unit calculates the minimum value of the clearance of the toe by using the position of the sensor in the advancing direction calculated by the sensor position calculation unit and the walking parameter at the timing at which the clearance of the toe is minimized. The determination unit verifies the minimum value of the clearance of the toe calculated by the calculation unit, and outputs a determination result based on the minimum value of the clearance of the toe.

In the present example embodiment, the determination result based on the minimum value of the clearance of the toe is output. According to the present example embodiment, it is possible to notify the user of the determination result based on the minimum value of the clearance of the toe.

### Fourth Example Embodiment

Next, a walking index calculation device according to a fourth example embodiment will be described with reference to the drawings. The walking index calculation device of the present example embodiment has a configuration in which the walking index calculation device of each example embodiment is simplified.

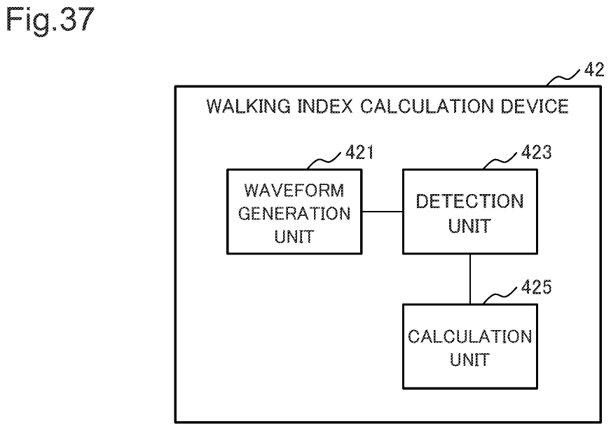

FIG. 37 is a block diagram illustrating an example of a configuration of a walking index calculation device 42 of the present example embodiment. The walking index calculation device 42 includes a waveform generation unit 421, a detection unit 423, and a calculation unit 425.

The waveform generation unit 421 generates a walking waveform by using sensor data regarding motion of a foot acquired by a sensor installed in footwear. The detection unit detects a timing at which the clearance of the toe is minimized from the walking waveform. The calculation unit 425 calculates the minimum value of the clearance of the toe by using a walking parameter at the timing at which the clearance of the toe is minimized.

The walking index calculation device of the present example embodiment calculates the minimum value of the clearance of the toe in walking of a user who lives a daily life by using the sensor data acquired by the sensor installed in the footwear. That is, according to the present example embodiment, the clearance of the toe can be calculated in walking in daily life.

(Hardware)

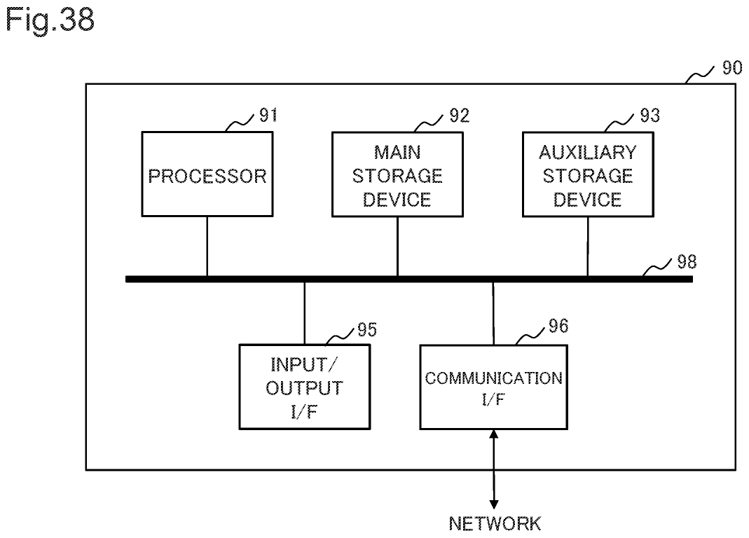

Here, a hardware configuration for executing the processing of the walking index calculation device according to each example embodiment of the present disclosure will be described by using an information processing apparatus 90 in FIG. 38 as an example. The information processing apparatus 90 in FIG. 38 is a configuration example for executing the processing of the walking index calculation device of each example embodiment, and does not limit the scope of the present disclosure.

As illustrated in FIG. 38, the information processing apparatus 90 includes a processor 91, a main storage device 92, an auxiliary storage device 93, an input/output interface 95, and a communication interface 96. In FIG. 38, the interface is abbreviated to I/F. The processor 91, the main storage device 92, the auxiliary storage device 93, the input/output interface 95, and the communication interface 96 are connected to each other via a bus 98 in such a way as to be capable of performing data communication. The processor 91, the main storage device 92, the auxiliary storage device 93, and the input/output interface 95 are connected to a network such as the Internet or an intranet via the communication interface 96.

The processor 91 loads a program stored in the auxiliary storage device 93 or the like into the main storage device 92 and executes the loaded program. In the present example embodiment, a software program installed in the information processing apparatus 90 may be used. The processor 91 executes processing of the walking index calculation device according to the present example embodiment.

The main storage device 92 has an area in which a program is loaded. The main storage device 92 may be a volatile memory such as a dynamic random access memory (DRAM). A nonvolatile memory such as a magnetoresistive random access memory (MRAM) may be configured and added as the main storage device 92.

The auxiliary storage device 93 stores various types of data. The auxiliary storage device 93 includes a local disk such as a hard disk or a flash memory. Various types of data may be stored in the main storage device 92, and the auxiliary storage device 93 may be omitted.

The input/output interface 95 is an interface for connecting the information processing apparatus 90 to a peripheral device. The communication interface 96 is an interface for connection to an external system or device via a network such as the Internet or an intranet based on a standard or a specification. The input/output interface 95 and the communication interface 96 may be shared as an interface connected to an external device.

An input device such as a keyboard, a mouse, or a touch panel may be connected to the information processing apparatus 90 as necessary. These input devices are used to input information and settings. In a case where the touch panel is used as an input device, a display screen of a display device may also serve as an interface of the input device. Data communication between the processor 91 and the input device may be relayed by the input/output interface 95.

The information processing apparatus 90 may be provided with a display device for displaying information. In a case where a display device is provided, the information processing apparatus 90 preferably includes a display control device (not illustrated) that controls display of the display device. The display device may be connected to the information processing apparatus 90 via the input/output interface 95.

The information processing apparatus 90 may be provided with a drive device. The drive device relays reading of data and a program from a recording medium, writing of a processing result of the information processing apparatus 90 to the recording medium, and the like between the processor 91 and the recording medium (program recording medium). The drive device may be connected to the information processing apparatus 90 via the input/output interface 95.

The above is an example of a hardware configuration for enabling the walking index calculation device according to each example embodiment of the present invention. The hardware configuration in FIG. 38 is an example of a hardware configuration for executing the arithmetic processing of the walking index calculation device according to each example embodiment, and does not limit the scope of the present invention. A program for causing a computer to execute processing related to the walking index calculation device according to each example embodiment is also included in the scope of the present invention. A program recording medium in which the program according to each example embodiment is recorded is also included in the scope of the present invention. The recording medium can be implemented by, for example, an optical recording medium such as a compact disc (CD) or a digital versatile disc (DVD). The recording medium may be implemented by a semiconductor recording medium such as a Universal Serial Bus (USB) memory or a secure digital (SD) card. The recording medium may be implemented by a magnetic recording medium such as a flexible disk or another recording medium. In a case where a program executed by the processor is recorded on a recording medium, the recording medium corresponds to a program recording medium.

The constituents of the walking index calculation device of each example embodiment can be freely combined. The constituents of the walking index calculation device of each example embodiment may be achieved by software or may be achieved by a circuit.

Although the present invention has been described with reference to the example embodiments, the present invention is not limited to the above example embodiments. Various modifications that can be understood by those skilled in the art can be made to the configuration and details of the present invention within the scope of the present invention.

### REFERENCE SIGNS LIST

1, 2, 3 walking index calculation system
11, 21, 31 data acquisition device
12, 22, 32, 42 walking index calculation device
111 acceleration sensor
112 angular velocity sensor
113 control unit
115 data transmission unit
121, 221, 321, 421 waveform generation unit
123, 223, 323, 423 detection unit
125, 225, 325, 425 calculation unit
224 sensor position calculation unit
327 determination unit

1. A walking index calculation device comprising:
a memory storing instructions, and
a processor connected to the memory and configured to execute the instructions to:
generate a walking waveform by using sensor data regarding motion of a foot acquired by a sensor installed in footwear worn by a user;
detect a timing at which a clearance of a toe is minimized from the walking waveform;
calculate a minimum value of the clearance of the toe by using a walking parameter at the timing at which the clearance of the toe is minimized;
determine a risk of falling of the user based on the minimum value of the toe clearance; and
cause a mobile terminal used by the user to vibrate in accordance with the timing at which the minimum value of the toe clearance is smaller than a threshold value for a fall risk.

2. The walking index calculation device according to claim 1, wherein
the processor is configured to execute the instructions to
calculate the minimum value of the clearance of the toe by using a value of a height of the sensor detected from a walking waveform of a vertical trajectory and a value of a rotation angle in a sagittal plane detected from a walking waveform of the rotation angle in the sagittal plane at the timing at which the clearance of the toe is minimized.

3. The walking index calculation device according to claim 2, wherein
the processor is configured to execute the instructions to
detect a timing of a gentle peak appearing between 40 and 60% of a gait cycle starting from a start timing of a support end stage in a walking waveform of an advancing direction acceleration as the timing at which the clearance of the toe is minimized.

4. The walking index calculation device according to claim 2, wherein
the processor is configured to execute the instructions to
detect a timing of zero crossing appearing between 40 and 60% of a gait cycle starting from a start timing of a support end stage in a walking waveform of a vertical acceleration as the timing at which the clearance of the toe is minimized.

5. The walking index calculation device according to claim 3, wherein
the processor is configured to execute the instructions to
calculate a first value by multiplying a sine of the rotation angle in the sagittal plane by a position of the sensor in an advancing direction at the timing at which the clearance of the toe is minimized,
calculate a second value by subtracting the first value from a height of the sensor at the timing at which the clearance of the toe is minimized, and
add a value of a height of the sensor at a timing of sole strike and the second value to calculate the minimum value of the clearance of the toe.

6. The walking index calculation device according to claim 5, wherein
the processor is configured to execute the instructions to
calculate the position of the sensor in the advancing direction by using a walking parameter at a timing of toe off detected from a walking waveform, and
calculate the minimum value of the clearance of the toe by using the position of the sensor in the advancing direction.

7. The walking index calculation device according to claim 2, wherein
the processor is configured to execute the instructions to
estimate the minimum value of the clearance of the toe by inputting the value of the height of the sensor and the value of the rotation angle in the sagittal plane at the timing at which the clearance of the toe is minimum into a machine learning model generated by machine learning with values of the vertical height and values of the rotation angle in the sagittal plane as explanatory variables and the minimum value of the toe clearance as the objective variable, and
display the determination result of the fall risk of the user according to the estimated minimum value of the toe clearance on the screen of the mobile terminal used by the user with content optimized for healthcare application.

8. A walking index calculation system comprising:
the walking index calculation device according to claim 1; and
a data acquisition device that measures spatial acceleration and spatial angular velocity, generates the sensor data based on the spatial acceleration and spatial angular velocity, and transmits the sensor data to the walking index calculation device.

9. An estimation method executed by a computer, the method comprising:
generating a walking waveform by using sensor data regarding motion of a foot acquired by a sensor installed in footwear worn by a user;
detecting a timing at which a clearance of a toe is minimized from the walking waveform;
calculating a minimum value of the clearance of the toe by using a walking parameter at the timing at which the clearance of the toe is minimized;
determining a risk of falling of the user based on the minimum value of the toe clearance; and
causing a mobile terminal used by the user to vibrate in accordance with the timing at which the minimum value of the toe clearance is smaller than a threshold value for a fall risk.

10. A non-transitory program recording medium recorded with a program causing a computer to perform the following processes:
generating a walking waveform by using sensor data regarding motion of a foot acquired by a sensor installed in footwear worn by a user;
detecting a timing at which a clearance of a toe is minimized from the walking waveform;
calculating a minimum value of the clearance of the toe by using a walking parameter at the timing at which the clearance of the toe is minimized;
determining a risk of falling of the user based on the minimum value of the toe clearance; and
causing a mobile terminal used by the user to vibrate in accordance with the timing at which the minimum value of the toe clearance is smaller than a threshold value for a fall risk.
